(19) United States
(12) Patent Application Publication
(10) Pub. No.: US 2006/0275893 A1
(43) Pub. Date: Dec. 7, 2006

(54) Biorelated substance examination apparatus and reaction stage thereof

(75) Inventors: Hidekazu Ishii, Hachioji-shi (JP); Takami Shibazaki, Hachioji-shi (JP); Hiroyuki Imabayashi, Hachioji-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS COROPORATION, TOKYO (JP)

(21) Appl. No.: 11/442,734

(22) Filed: May 26, 2006

Related U.S. Application Data
(63) Continuation of application No. PCT/JP03/15263, filed on Nov. 28, 2003.

(51) Int. Cl. C12M 1/34; G02B 21/34

(52) U.S. Cl. 435/288.7; 435/303.1; 435/809; 359/395; 359/398

(57) ABSTRACT

A biorelated substance examination apparatus includes a reaction stage which supports a reaction vessel which contains the solid support and promotes a reaction of the solid-state phase, and a microscope which optically observes the solid support. The reaction stage includes a fluid transfer mechanism which transfers a fluid with respect to the reaction vessel and a temperature adjusting mechanism which adjusts a temperature of the solid support in the reaction vessel.

### CROSS-REFERENCE TO RELATED APPLICATIONS

This is a Continuation Application of PCT Application No. PCT/JP03/15263, filed Nov. 28, 2003, which was published under PCT Article 21(2) in Japanese.

### BACKGROUND OF THE INVENTION

1. Field of the Invention

The present invention relates to a biorelated substance examination apparatus and a reaction stage.

2. Description of the Related Art

Recently, with advances in gene analysis techniques, the gene sequences of many creatures including human beings have been revealed. In addition, the causal relationships between analyzed gene products and diseases have been gradually clarified.

A currently used gene examination method comprises a step of extracting a nucleic acid from a biological sample, a step of amplifying a target gene as an examination target by using a nucleic acid amplifying method called the PCR or NASBA method or the like, and a step of labeling the nucleic acid with a radioactive isotope, a fluorescent molecule, or the like, and a step of measuring the base sequence or concentration of the labeled target gene.

Recently, a capillary electrophoresis method has widely been used, which can quickly process many fluorescence-labeled nucleic acids as samples by using a plurality of capillaries. This makes it possible to perform such process within a period of time approximately ⅓ to ¼ that in a method using a conventional electrophoresis apparatus or the like.

Furthermore, an examination method using DNA chips for simultaneously examining a plurality of genes has recently been developed. DNA chips include a DNA chip having many cDNA probes immobilized on the surface of a glass substrate, and a DNA chip having many oligo probes synthesized in small regions on a silicon wafer by applying a semiconductor manufacturing process. In any case, pluralities of base sequences and expression amount of DNAs in a sample can be simultaneously determined.

Using DNA chips makes it possible to simultaneously analyze many gene expression amounts and a plurality of mutations. In addition, many genes can be classified into a plurality of groups (i.e., clustering) according to the data obtained by using DNA chips, and information about variations in genes accompanying development and differentiation can be also be obtained. The gene information obtained in this manner is used as a database like that which can be easily accessed through the Internet.

Conventionally, the electrophoresis method and microarray method have been used for the examination of the expression amounts of genes and the analysis of mutations. However, the electrophoresis method requires much time for examination. In addition, the number of examinations that can be performed at once is limited to a small number.

The gene analysis method using DNA chips can perform many examinations at once. However, this method partly demands a long examination time, includes many examination steps, and requires complicated operation. For this reason, analysis results with high repeatability cannot be obtained.

In order to overcome such drawbacks, a technique using a porous glass wafer manufactured by using a microfabrication process as a DNA chip carrier has been developed. A technique using such a porous glass wafer as a DNA chip carrier has been disclosed in, for example, PCT (WO) 9-504864. With this technique, examination similar to that using a DNA chip can be performed with high repeatability within a short period of time.

In the conventional technique using a porous glass wafer as a DNA chip carrier, a reaction portion which performs movement of liquid and temperature control is separate from a measurement portion which performs fluorescence detection. This requires various types of apparatus arrangements. For this reason, it takes much time and labor for transportation between the respective types of apparatuses. In addition, for example, temperature changes occur during transportation and the problem of the adhesion of dust occurs due to transportation. That is, much attention is needed to ensure the repeatability of measurement results.

In clinical examination, in particular, strong demands have arisen for easy examination of a gene with high accuracy within a short period of time. It is thought to be difficult to meet these user needs.

### BRIEF SUMMARY OF THE INVENTION

It is another object of the present invention to provide a reaction stage which is suitably used for such an examination apparatus.

The present invention is, in one aspect, a biorelated substance examination apparatus for examining a reaction of a biorelated substance using a solid support which allows a fluid to pass therethrough. The apparatus includes a reaction stage for supporting a reaction vessel which contains a solid support and promoting a reaction of the solid-state phase, and a microscope for optically observing the solid support. The reaction stage includes a fluid transfer mechanism for transferring a fluid with respect to the reaction vessel, and a temperature adjusting mechanism for adjusting a temperature of the solid support in the reaction vessel.

The present invention is, in another aspect, a reaction stage for an apparatus which examines a reaction of a biorelated substance using a solid support which allows a fluid to pass therethrough. The stage includes a reaction vessel support mechanism which supports a reaction vessel which contains a solid support, a fluid transfer mechanism for transferring a fluid with respect to the reaction vessel, and a temperature adjusting mechanism for adjusting a temperature of the solid support in the reaction vessel.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic perspective view of a gene examination apparatus according to the first embodiment of the present invention, showing a state wherein a reaction stage is drawn out;

FIG. 2 is a schematic perspective view of the gene examination apparatus according to the first embodiment of the present invention, like FIG. 1, illustrating a hood in transparent to show a reaction stage housed in the hood;

FIG. 3 is a perspective view of the reaction vessel shown in FIGS. 1 and 2, illustrating both an assembled state and a disassembled state of the reaction vessel;

FIG. 4 is an enlarged perspective view of the slide chip shown in FIG. 3;

FIG. 5 is an exploded perspective view of the slide chip shown in FIG. 4;

FIG. 6 is a view showing the upper surface, a longitudinal section, and a cross-section of the reaction vessel shown in FIG. 3;

FIG. 7 is a view showing many nucleic acid probe spots arrayed on a DNA microarray;

FIG. 8 is a perspective view of the reaction stage shown in FIGS. 1 and 2, showing a state before the reaction vessel is mounted;

FIG. 9 is a perspective view of the reaction stage shown in FIGS. 1 and 2, showing a state after the reaction vessel is mounted;

FIG. 10 is a sectional view of the reaction stage and reaction vessel shown in FIG. 9, showing a state wherein a nucleic acid sample solution is located on a DNA microarray immediately after the reaction vessel is mounted;

FIG. 11 is a sectional view of the reaction stage and reaction vessel shown in FIG. 9, showing a state wherein a nucleic acid sample solution is transferred into a liquid storage hole;

FIG. 12 is a view schematically showing the channels of the gene examination apparatus according to the first embodiment of the present invention;

FIG. 13 is a view schematically showing the temperature adjusting mechanism and its control system of the gene examination apparatus according to the first embodiment of the present invention;

FIG. 14 is a view schematically showing the arrangement of a syringe piston which can be used in place of the syringe piston shown in FIGS. 10 and 11;

FIG. 15 is a sectional view of a reaction stage and a reaction vessel in the second embodiment of the present invention, showing a state wherein a DNA microarray is cleaned by ultrasonic cleaning using piezoelectric elements provided for a base portion;

FIG. 16 is a sectional view of the reaction stage, reaction vessel, and cleaning nozzle of a gene examination apparatus according to the third embodiment of the present invention, showing a state wherein washing water is supplied from the cleaning nozzle;

FIG. 17 is a sectional view of the reaction stage, reaction vessel, and cleaning nozzle of the gene examination apparatus according to the third embodiment of the present invention, showing a state wherein washing water is sucked by the cleaning nozzle;

FIG. 18 is a sectional view of the reaction stage and reaction vessel of a gene examination apparatus according to the fourth embodiment of the present invention, showing a state wherein a nucleic acid sample solution is positioned on a DNA microarray;

FIG. 19 is a sectional view of the reaction stage and reaction vessel of a gene examination apparatus according to the fourth embodiment of the present invention, showing a state wherein a nucleic acid sample solution is transferred into a liquid storage hole;

FIG. 20 is a view schematically showing the channels of the gene examination apparatus according to the fourth embodiment of the present invention;

FIG. 21 is a sectional view of the reaction vessel, cleaning nozzle, and waste liquid nozzle of a gene examination apparatus according to the fifth embodiment of the present invention;

FIG. 22 is a sectional view of a reaction vessel according to the sixth embodiment of the present invention;

FIG. 23 is a schematic perspective view of a gene examination apparatus according to the seventh embodiment of the present invention;

FIG. 24 is a sectional view of the reaction stage, reaction vessel, cleaning nozzle, and waste liquid nozzle of a gene examination apparatus according to the eighth embodiment of the present invention, showing a state wherein washing water is supplied from the cleaning nozzle;

FIG. 25 is a sectional view of the reaction stage, reaction vessel, cleaning nozzle, and waste liquid nozzle of the gene examination apparatus according to the eighth embodiment of the present invention, showing a state wherein washing water is sucked by the waste liquid nozzle;

FIG. 26 is a sectional view of the reaction stage, reaction vessel, cleaning nozzle, and waste liquid nozzle of the gene examination apparatus according to the eighth embodiment of the present invention, showing a state wherein washing water is supplied from a syringe piston pump;

FIG. 27 is a sectional view of the reaction stage, reaction vessel, cleaning nozzle, and waste liquid nozzle of the gene examination apparatus according to the eighth embodiment of the present invention, showing a state wherein washing water is sucked by the waste liquid nozzle;

FIG. 28 is a view schematically showing the channels of the gene examination apparatus according to the eighth embodiment of the present invention;

FIG. 29 is a sectional view of the reaction stage, reaction vessel, cleaning nozzle, and waste liquid nozzle of a gene examination apparatus according to the ninth embodiment of the present invention, showing a state wherein washing water is supplied from the cleaning nozzle;

FIG. 30 is a sectional view of the reaction stage, reaction vessel, cleaning nozzle, and waste liquid nozzle of the gene examination apparatus according to the ninth embodiment of the present invention, showing a state wherein washing water is sucked by the waste liquid nozzle;

FIG. 31 is a view schematically showing the channels of a gene examination apparatus according to the ninth embodiment of the present invention;

FIG. 32 is a sectional view of the reaction stage and reaction vessel of a gene examination apparatus according to the tenth embodiment of the present invention;

FIG. 33 is a sectional view of the reaction stage and reaction vessel of the gene examination apparatus according to the tenth embodiment of the present invention, showing a state wherein a nucleic acid sample solution is positioned on a DNA microarray;

FIG. 34 is a sectional view of the reaction stage, reaction vessel, and cleaning nozzle of the gene examination apparatus according to the tenth embodiment of the present invention, showing a state wherein washing water is supplied from the cleaning nozzle;

FIG. 35 is a sectional view of the reaction stage and reaction vessel of the gene examination apparatus according to the tenth embodiment of the present invention, showing a state wherein washing water is discharged by the waste liquid hole;

FIG. 36 is a sectional view of the reaction stage, reaction vessel, and waste liquid nozzle of the gene examination apparatus according to the tenth embodiment of the present invention, showing a state wherein washing water is sucked by the waste liquid nozzle;

FIG. 37 is a sectional view of the reaction stage and reaction vessel of a gene examination apparatus according to the eleventh embodiment of the present invention; and

FIG. 38 is a view showing a modification of the reaction stage shown in FIGS. 10 and 11.

### DETAILED DESCRIPTION OF THE INVENTION

The embodiments of the present invention will be described below with reference to the views of the accompanying drawing.

### First Embodiment

The first embodiment of the present invention is directed to an apparatus for examining a gene as a biorelated substance and, more particularly, to a gene examination apparatus which uses a DNA microarray.

The basic principle of a detection method executed in a gene examination apparatus according to this embodiment is that a nucleic acid chain having a specific sequence which is contained in a sample is detected by using an immobilized nucleic acid probe with a known sequence. In this method, for example, single-stranded nucleic acids probes are immobilized on a substrate, and are brought into contact with the fluorescence-labeled nucleic acid contained in the sample. If the nucleic acid in the sample has a sequence complementary to the nucleic acid probe, the nucleic acid in the sample hybridizes with the nucleic acid probe to form a double-stranded structure, which is immobilized on the substrate. Therefore, fluorescence-detecting the label (Rhodamine, FITC, Cy3, Cy5, or the like) attached to the probe detects the target nucleic acid having a sequence complementary to the probe. In addition, cleaning off unreacted nucleic acids increases the S/N ratio of fluorescence detection.

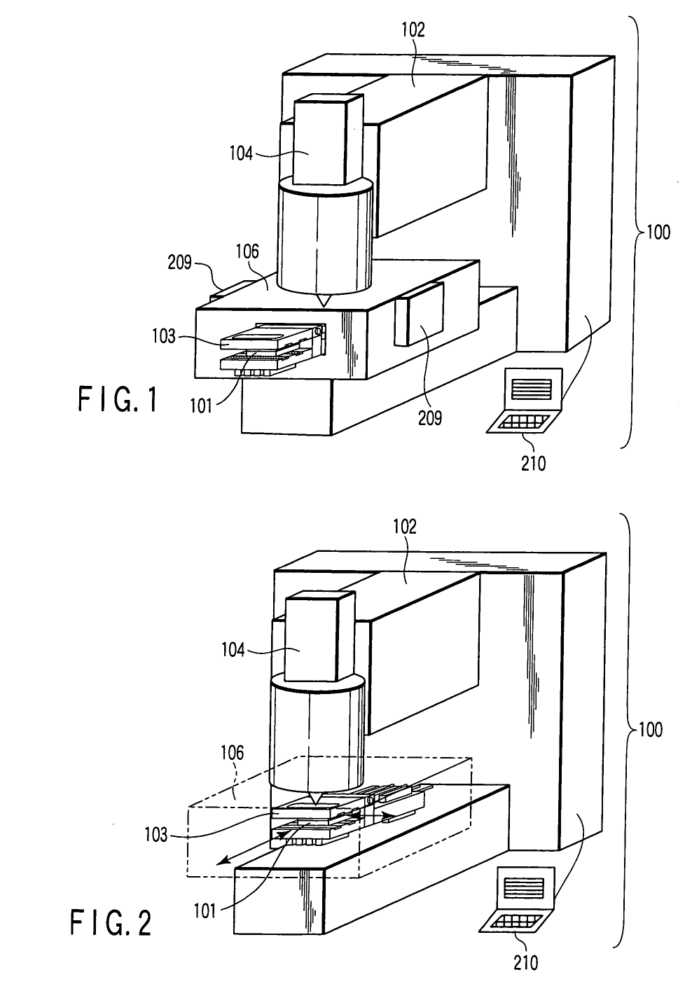

As shown in FIGS. 1 and 2, a gene examination apparatus 100 in this embodiment comprises a reaction stage 103 for supporting a reaction vessel 101 which contains a DNA microarray and promoting a reaction of the DNA microarray, and a microscope 102 for optically observing the DNA microarray. The microscope 102 has an X-Y stage, on which the reaction stage 103 is mounted. The reaction stage 103 is moved by the X-Y stage to, for example, change the field of view observed by the microscope 102.

A control computer 210 is for controlling the operation of the gene examination apparatus 100 in accordance with a predetermined examination step and performing data processing, such as storing, computing, and displaying examination results.

The gene examination apparatus 100 further comprises a CCD (charge coupled device) camera 104 for sensing a fluorescence image of the DNA microarray which is obtained by the microscope 102. The CCD camera 104 is preferably a cooled type for the sake of an improvement in examination accuracy.

The gene examination apparatus 100 further includes a hood 106 for covering the reaction stage 103. The hood 106 is provided in order to block disturbance light unnecessary for microscopic observation, prevent variations in temperature environment, and prevent the intrusion of dust into the observation area. The hood 106 has an inserting and drawing port for inserting and drawing out the reaction stage 103 and an observation window which allows microscopic observation.

The reaction stage 103 is drawn out from the hood 106 by the X-Y stage as shown in FIG. 1 when the reaction vessel 101 is to be placed. At the time of observation, as shown in FIG. 2, the reaction stage 103 is housed in the hood 106. The inserting and drawing port of the hood 106 has an opening/closing door, which is closed after the reaction stage 103 is housed in the hood 106. The environment in the hood 106 may be kept constant by a temperature adjusting device or humidity adjusting device provided in the hood 106.

The gene examination apparatus 100 comprises a cooling mechanism for lowering the temperature of a solid support in the reaction vessel 101 by forced convection. The cooling mechanism includes blowers 209 which feed air to the reaction stage 103 through the ventilating holes formed in the hood 106. More preferably, the blowers 209 include Peltier elements for cooling air fed to the reaction stage 103. FIG. 1 shows the two blowers 209, but the number and positions of blowers are not limited to these and can be arbitrarily changed.

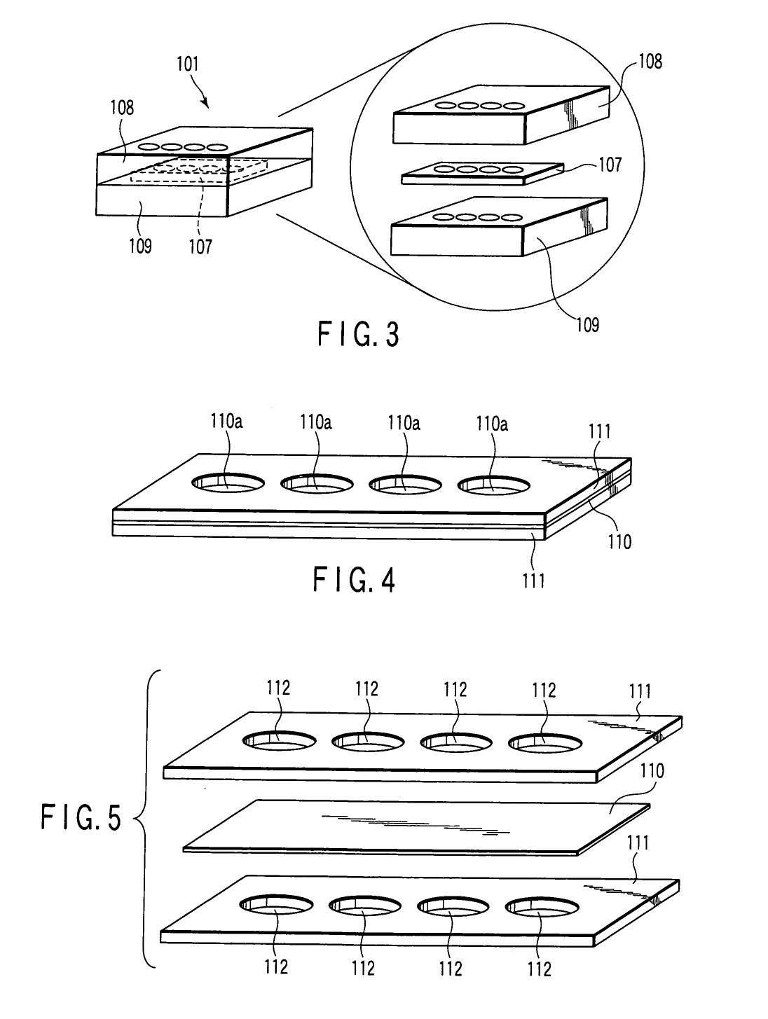

As shown in FIG. 3, the reaction vessel 101 comprises a slide chip 107 in the form of a thin plate which has four DNA microarrays 110a and upper and lower housings 108 and 109 which hold the slide chip 107 by vertically sandwiching it. The upper housing 108 and the lower housing 109 preferably comprise light-shielding members, and more preferably comprise dark members. The upper housing 108 and the lower housing 109 hold the slide chip 107 by fixing each other with a fastening means such as screws or an adhesive. O-rings (not shown), which are provided on the upper and lower surfaces of the slide chip 107 so as to surround the four DNA microarrays 110a, prevent a fluid (a nucleic acid sample solution or a cleaning fluid) to be supplied later from leaking through the gap between the upper housing 108 and the lower housing 109. The members for preventing the leakage of fluid are not limited to O-rings, and an arbitrary seal can be used as long as the leakage of liquid can be prevented. For example, a member for preventing the leakage of fluid may be one rubber sheet which covers the entire upper or lower surface of the slide chip 107 except for the four DNA microarrays 110a. Alternatively, an adhesive which can provide sealing may be used.

Although the reaction vessel 101 shown in FIG. 3 includes the four DNA microarrays 110a, the number of DNA microarrays 110a included in one reaction vessel 101 is not limited to this, and may be arbitrarily changed.

As shown in FIGS. 4 and 5, the slide chip 107 comprises, for example, a ceramic porous portion 110 as the solid support and a pair of support plates 111 for supporting the porous portion 110 by sandwiching it. The pair of support plates 111 each have four opening portions 112 for defining the DNA microarrays 110a at corresponding positions. That is, one DNA microarray 110a is a portion of the porous portion 110 which is exposed through the pair of opening portions 112 located on and under the porous portion 110.

The porous portion 110 has many minute holes extending in the thickness direction. Any material can be used for the porous portion 110 as long as the porous portion 110 does not break down at the temperature and pressure to which the porous portion 110 is exposed at the time of a gene reaction.

The porous portion 110 may have any structure as long as a liquid can pass therethrough.

If the porous portion 110 is manufactured so that it has a constant thickness and all holes become identical, all the holes have an almost equal capacity. In addition, if the thickness of the porous portion 110 is sufficiently decreased and the diameters of the holes are sufficiently decreased, desired amounts of sample and reagent can be distributed and stored in many through holes. If, therefore, a necessary number of holes for the storage of a desired amount of liquid are formed per a specific unit area on the surface of the porous portion 110, and a sample or a reagent is supplied to a predetermined area of the porous portion 110, stable analysis can be executed in the porous portion 110.

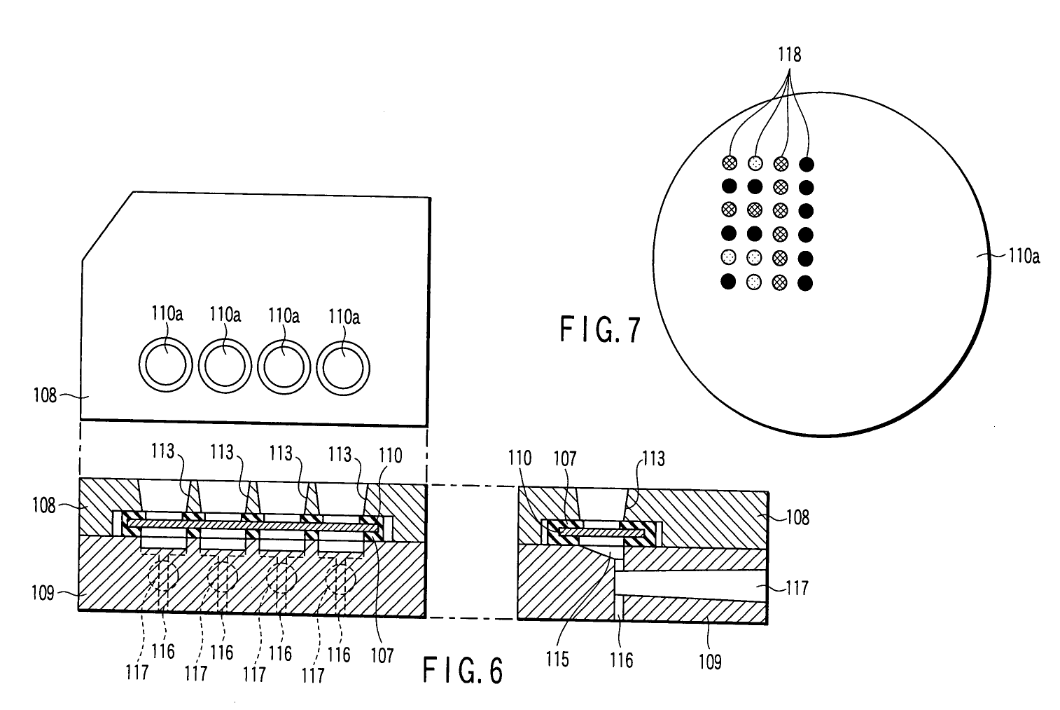

As shown in FIG. 6, the upper housing 108 has four tapered opening portions 113 for exposing the four DNA microarrays 110a of the slide chip 107. A fluid containing a nucleic acid sample to be supplied to the DNA microarray 110a can be stored in the inner space of the tapered opening portion 113.

The lower housing 109 includes four recess portions 115 which have inclined bottom surfaces and are located at positions corresponding to the four DNA microarrays 110a of the slide chip 107, waste liquid holes 116 for waste liquid, which extend from the lowermost portion of the recess portions 115 to the lower surface of the lower housing 109, and liquid storage holes 117 which extend midway from the waste liquid holes 116 to a side surface of the lower housing 109 and can store a fluid containing the nucleic acid sample to be supplied to the DNA microarrays 110a.

A plurality of droplets of a nucleic acid probe solution are discharged to the porous portion 110 of each DNA microarray 110a by a liquid dispensing device using an inkjet discharge principle. As shown in FIG. 7, one droplet of a nucleic acid probe solution which has discharged to the porous portion 110 is instantly sucked in fine holes to form a nucleic acid probe spot 118 with a small diameter.

One DNA microarray 110a has many nucleic acid probe spots 118 formed in the porous portion 110. In general, one nucleic acid probe spot 118 includes one type of nucleic acid probe, which is immobilized to the inner wall of fine holes in part of the porous portion 110 included in the spot. In other words, an area including fine holes to which one type of nucleic acid probe is immobilized is one nucleic acid probe spot 118.

In general, different types of nucleic acid probes are immobilized to different nucleic acid probe spots 118 in one DNA microarray 110a. However, identical nucleic acid probes may be immobilized to a plurality of nucleic acid probe spots 118 in one DNA microarray 110a, as needed. In addition, frictional resistance with respect to a fluid or the adsorptivity of a reagent such as the nucleic acid probe may be adjusted by performing surface treatment for the fine-porous inner wall of the porous portion 110.

The size of the slide chip 107 is, for example, ½ that of a general slide glass, and is approximately 37 mm×approximately 12 mm×approximately 1 mm. For example, one DNA microarray 110a has a diameter of approximately 4 mm, one nucleic acid probe spot 118 a diameter of approximately 100 μm. Four hundred nucleic acid probe spots 118 are arrayed at 200-μm intervals in one DNA microarray 110a.

One nucleic acid probe spot 118 has 100 to 1,000 fine holes, more preferably, 1,000 fine holes. One hole has a diameter of 0.05 to 5 μm.

One DNA microarray 110a corresponds to one reaction unit for the execution of one type of reaction. A conventional DNA chip is formed by immobilizing a probe to a slide glass surface, and has a reaction area with a two-dimensional spread. Unlike such a DNA chip, the DNA microarray 110a has a three-dimensional reaction area having a spread in the thickness direction in addition to a two-dimensional spread on a substrate surface.

The nucleic acid probe is generally a polynucleotide or oligonucleotide comprising approximately 10 nucleotides or more or approximately 100 nucleotides or less, and is generally used to detect a nucleic acid by hybridization.

One nucleic acid probe spot 118 contained in the DNA microarray 110a has a plurality of fine holes, i.e., three-dimensional spaces which can store a small amount of liquid, as described above. One nucleic acid probe spot 118 has three-dimensional spaces which are very small within a plane when viewed from above and extend in the thickness direction of the porous portion 110. Therefore, the area actually occupied by one nucleic acid probe spot 118 is much larger than that seen.

For example, many pieces of information can be obtained with sufficient sensitivity in one small DNA microarray 110a by changing conditions (e.g., changing the type of nucleic acid probe to be immobilized and changing surface treatment) for each nucleic acid probe spot 118.

The four DNA microarrays 110a included in the slide chip 107 may be applied to the examination of different samples (nucleic acid sample solutions 114) or may be applied to four different types of analyses concerning one sample.

Analysis using the DNA microarray 110a includes, for example, the detection of the presence/absence of mutations such as cancer-related genes and drug metabolic genes, and polymorphs, and the expression analysis of cancer-related genes, apoptosis-related genes, and the like.

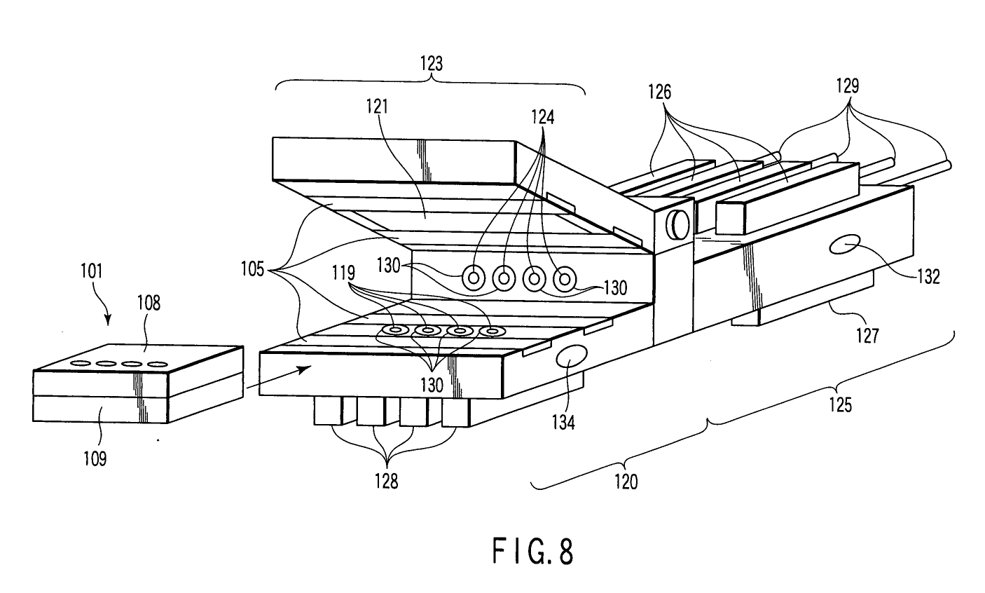

The reaction stage 103 for supporting the reaction vessel 101 and promoting a reaction will be described with reference to FIGS. 8 to 11.

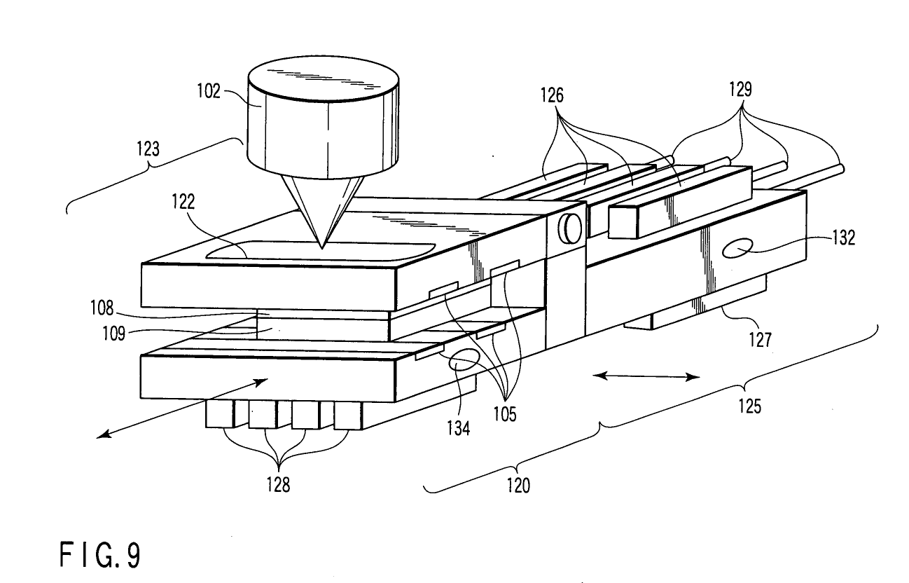

As shown in FIGS. 8 and 9, the reaction stage 103 includes a base portion 120 on which the reaction vessel 101 is placed, and a cover portion 123 which can open and close with respect to the base portion 120. The base portion 120 and the cover portion 123 constitute a reaction vessel support mechanism which supports the reaction vessel 101.

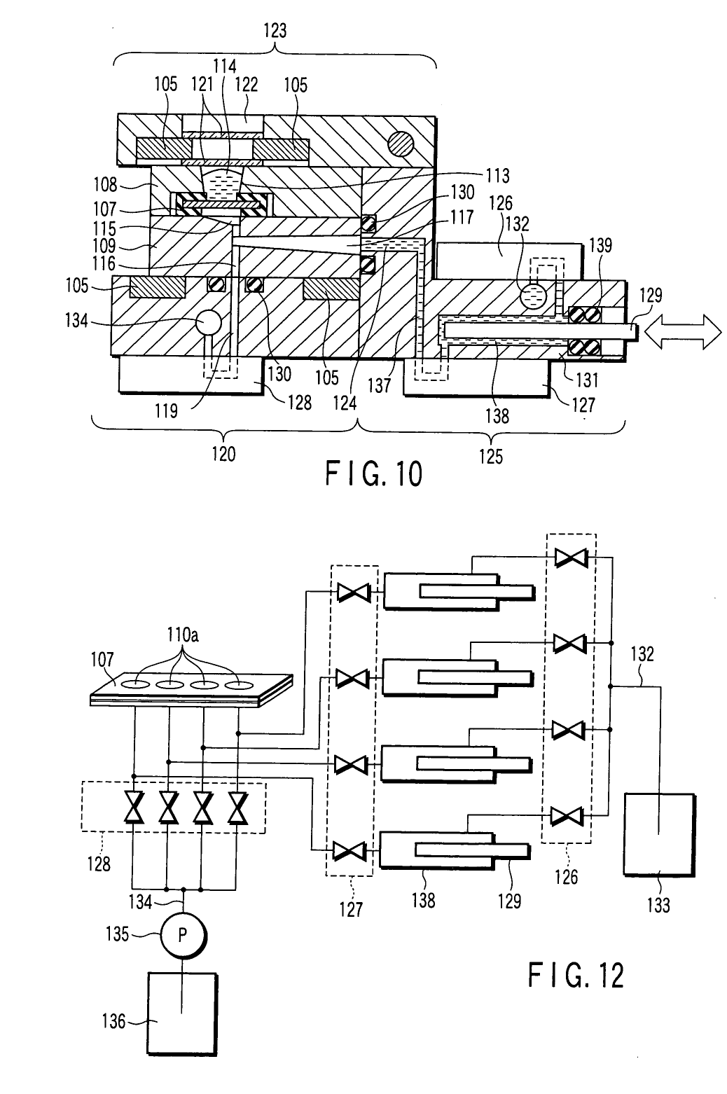

The cover portion 123 includes a microscope observation hole 122 which allows the observation of the DNA microarray 110a with the microscope 102, and two optically transparent cover glasses 121 which close the microscope observation hole 122. As shown in FIGS. 10 and 11, the two cover glasses 121 are spaced apart from each other.

The base portion 120 includes four discharge channels 119 each of which is fluidally communicated with a corresponding one of the waste liquid holes 116 of the reaction vessel 101, and four electromagnetic valves 128 which are provided midway along the respective discharge channels 119 to open and close them. All the four discharge channels 119 are fluidally in communication with a waste liquid collecting pipe 134. The waste liquid collecting pipe 134 terminates at a side surface of the base portion 120. However, the waste liquid collecting pipe 134 need not always terminate at a side surface of the base portion 120. For example, the waste liquid collecting pipe 134 may terminate at an upper or lower surface of the base portion 120. The waste liquid collecting pipe 134 preferably terminates at the lower surface because the flow of waste liquid from the base portion 120 to the waste liquid collecting pipe 134 is made smooth by gravitation.

Discharge channels 119 terminate at a surface facing the reaction vessel 101, circular grooves are formed around the terminations, i.e., the liquid transfer ports, and O-rings 130 are placed in the grooves. The discharge channels 119 are fluidally communicated with the waste liquid holes 116 of the reaction vessel 101 supported on the reaction stage 103 in a liquid-tight fashion by the O-rings 130. As long as the liquid-tight fashion is kept between the discharge channels 119 and the waste liquid holes 116, the O-rings 130 and the circular grooves around the liquid transfer ports may be provided for the reaction vessel 101.

The reaction stage 103 further includes a syringe piston pump unit 125 which is a fluid transfer mechanism for transferring a fluid with respect to the reaction vessel 101. The syringe piston pump unit 125, i.e., the fluid transfer mechanism, moves a nucleic acid sample solution supplied into the reaction vessel 101 within the reaction vessel 101, and supplies washing water into the reaction vessel 101.

The syringe piston pump unit 125 includes four transfer channels 124 which are fluidally communicated with the respective liquid storage holes 117 of the reaction vessel 101, respectively, and four syringe piston pumps 131 which are transfer pumps, which are fluidally in communication with the respective transfer channels 124, respectively, for transferring the fluid in the reaction vessel 101. The syringe piston pump unit 125 further includes electromagnetic valves 127 which are provided midway along the transfer channels 124 to open and close the transfer channels 124.

Transfer channels 124 terminate at a surface facing the reaction vessel 101, circular groove are formed around the terminations, i.e., the liquid transfer ports, and O-rings 130 are placed in the grooves. The transfer channels 124 are fluidally communicated with the liquid storage holes 117 of the reaction vessel 101 supported on the reaction stage 103 in a liquid-tight fashion by the O-rings 130. As long as the liquid-tight fashion is kept between the transfer channels 124 and the liquid storage holes 117, the O-rings 130 and the circular grooves around the liquid transfer ports may be provided in the reaction vessel 101.

Each syringe piston pump 131 includes a syringe 138, a piston 129 which can move in the syringe 138, a seal 139 for keeping the syringe 138 and the piston 129 in a liquid-tight state. The seal 139 is, for example, an O-ring fixed to the syringe 138. However, the seal 139 is not limited to an O-ring, and another arbitrary proper seal, e.g., a packing or a Teflon seal, may be used.

The piston 129 of the syringe piston pump unit 125 is driven by, for example, a motor unit (not shown). In other words, although not shown, the gene examination apparatus 100 includes motor units for driving the pistons 129 of the syringe piston pump unit 125. Each motor unit comprises, for example, a stepping motor and a rack-pinion mechanism which converts the motion of the rotating shaft of the stepping motor into linear motion.

The gene examination apparatus 100 may include one motor unit which simultaneously drives the four pistons 129 of the syringe piston pump unit 125. More preferably, however, the gene examination apparatus 100 includes four motor units for independently driving the four pistons 129 of the syringe piston pump unit 125.

The syringe piston pump unit 125 further includes four channels extending from the respective syringe piston pumps.131 and four electromagnetic valves 126 which are provided midway along the respective channels to open and close them. All the four channels are fluidally in communication with a collecting piping 132. The collecting piping 132 terminates at a side surface of the syringe piston pump unit 125. Note, however, that the collecting piping 132.need not always terminate at a side surface of the syringe piston pump unit 125. For example, the collecting piping 132 may terminate at the upper or lower surface of the pump unit 125 and, more preferably, terminate at a side surface because the channels are simplified and become easy to process.

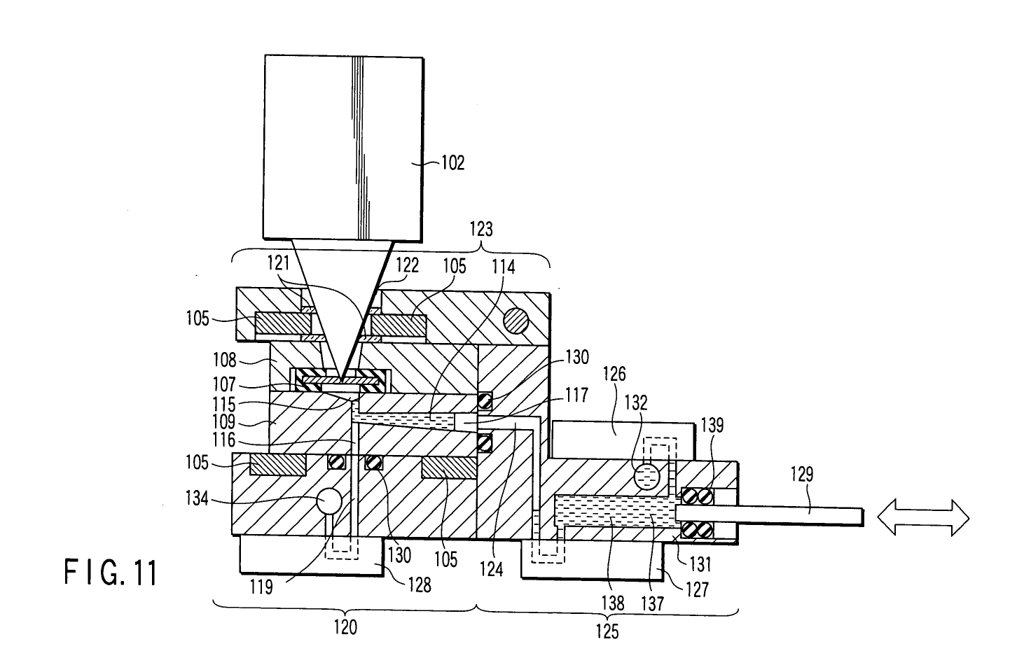

As schematically shown in FIG. 12, the collecting piping 132 of the syringe piston pump unit 125 is fluidally communicated with a washing water tank 133. The waste liquid collecting pipe 134 provided on the base portion 120 is fluidally communicated with the waste liquid tank 136 through a suction pump 135. In other words, the gene examination apparatus 100 further includes the washing water tank 133 which is fluidally in communication with the syringe piston pump unit 125 of the reaction stage 103 and a waste liquid tank 136 which is fluidally in communication with the discharge channel 119 of the reaction stage 103.

Referring to FIG. 12, for example, the electromagnetic valve 127 on the DNA microarray 110a side and the electromagnetic valve 128 on the waste liquid channel side are closed, the electromagnetic valve 126 on the washing water tank 133 side is opened, and the piston 129 of the syringe piston pump 131 is moved to the suction side (the right side with respect to the drawing surface), so that washing water 137 in the washing water tank 133 is sucked into the syringe 138. Then the electromagnetic valve 126 on the washing water tank 133 is closed, the electromagnetic valve 127 on the DNA microarray 110a side is opened, and the piston 129 is moved to the discharge side (the left side with respect to the drawing surface), so that the washing water 137 is fed to each porous portion 110 of the DNA microarray 110a.

In another operation, the electromagnetic valve 126 on the washing water tank 133 side and the electromagnetic valve 128 on the waste liquid channel side are closed, the electromagnetic valve 127 on the DNA microarray 110a side is opened, and the piston 129 is repeatedly moved to the suction side and the discharge side, so that the nucleic acid sample solution 114 stored in the reaction vessel 101 is made to repeatedly pass through the DNA microarray 110a while being agitated.

In still another operation, the electromagnetic valve 128 on the waste liquid channel side is opened, the electromagnetic valve 127 on the DNA microarray 110a is closed, and the suction pump 135 is operated, so that the nucleic acid sample solution 114 and washing water 137 on each porous portion 110 are discharged to the waste liquid tank 136.

By selectively driving the four electromagnetic valves 126, the four electromagnetic valves 127, and the four electromagnetic valves 128, an arbitrary one of the four DNA microarrays 110a can be selectively used for examination.

In addition, by selectively driving the four motor units provided for the respective pistons 129 of the syringe piston pump unit 125, an arbitrary one of the four DNA microarrays 110a can be selectively used for examination.

Pressure sensors for detecting the pressure of a fluid flowing in channels may be provided midway along the channels which communicate between the reaction vessel 101 and the syringe piston pumps 131 or midway along the channels which communicate between the reaction vessel 101 and the suction pumps 135. In other words, the syringe piston pump unit 125 may include pressure sensors provided midway along the transfer channels 124. In addition, the gene examination apparatus 100 may have pressure sensors midway along the channels which communicate between the reaction vessel 101 and the suction pumps 135.

For example, by monitoring the pressures of fluid using each pressure sensor, the syringe piston pump 131 and the suction pump 135 may be controlled such that the pressure of the fluid does not exceed a predetermined pressure.

After the start of the operation of the syringe piston pump 131 and suction pump 135, a numerical value equal to or higher than a predetermined pressure may be monitored by the pressure sensor to quantitatively detect that the porous portion 110 has been clogged before the start of the operation of the syringe piston pump 131 and the suction pump 135.

After the start of the operation of the syringe piston pump 131 and suction pump 135, a numerical value equal to or higher than a predetermined pressure may be monitored by the pressure sensor to quantitatively detect that the porous portion 110 has been clogged with a foreign substance during the operation of the syringe piston pump 131 and suction pump 135.

After the start of the operation of the syringe piston pump 131 and suction pump 135, a numerical value equal to or lower than a predetermined pressure may be monitored by the pressure sensor to quantitatively detect that the porous portion 110 has cracked before the start of the operation of the syringe piston pump 131 and suction pump 135.

After the start of the operation of the syringe piston pump 131 and suction pump 135, a numerical value equal to or lower than a predetermined pressure may be monitored by the pressure sensor to quantitatively detect that a leak has occurred between the reaction stage 103 and the DNA microarray 110a.

After the start of the operation of the syringe piston pump 131 and suction pump 135, a numerical value within a predetermined pressure range may be monitored by the pressure sensor to quantitatively detect and confirm that the sucking operation and discharging operation of the syringe piston pump 131 and the sucking operation of the suction pump 135 are normally performed.

After the start of the operation of the syringe piston pump 131 and suction pump 135, a numerical value within a predetermined pressure range may be monitored by the pressure sensor to quantitatively detect that the nucleic acid sample solution 114 or washing water 137 has been dispensed or supplied to the porous portion 110.

The reaction stage 103 further includes a temperature adjusting mechanism for adjusting the temperature of the DNA microarrays 110a in the reaction vessel 101. The temperature adjusting mechanism includes heating means for raising the temperature of the reaction vessel 101. The heating means are, for example, plate-like heaters 105 such as silicone rubber heaters, ceramic heaters, or Peltier elements, as shown in FIGS. 8 to 11.

The plate-like heaters 105, which are provided for the base portion 120 and the cover portion 123, preferably come into surface contact with the reaction vessel 101 supported on the reaction stage 103. More specifically, as shown in FIGS. 10 and 11, the plate-like heaters 105 provided for the base portion 120 come into surface contact with the lower housing 109 of the reaction vessel 101 placed on the base portion 120.

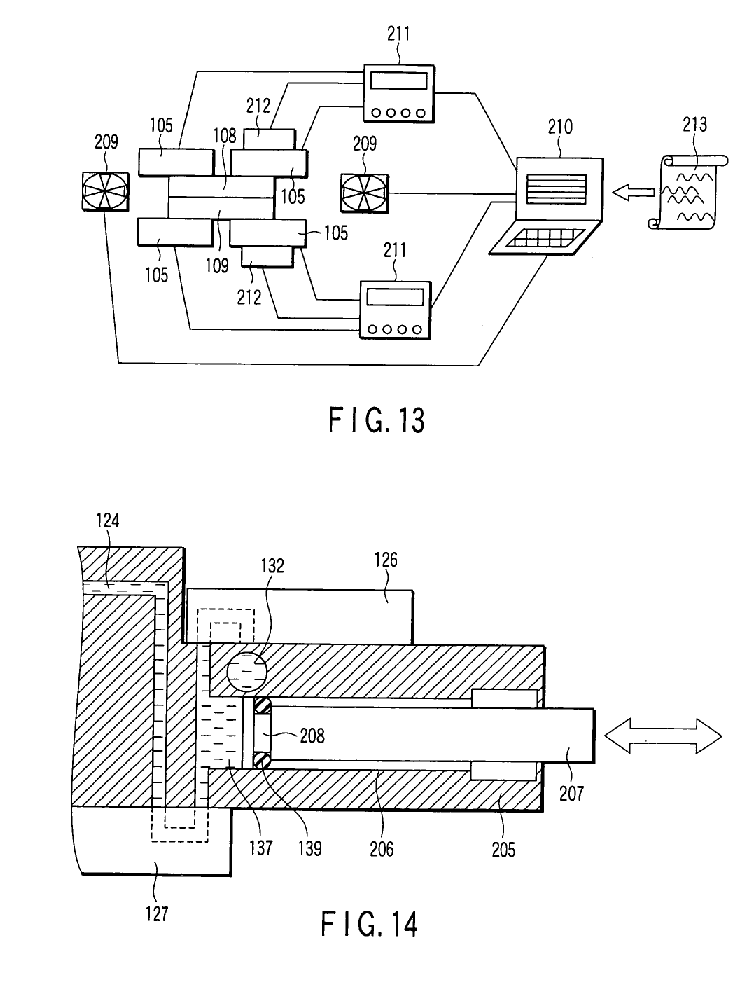

As shown in FIG. 13, the plate-like heaters 105 are in electrical connection with temperature adjusting devices 211, and are driven and controlled by the temperature adjusting devices 211. The temperature adjusting devices 211 are in electrical connection with the control computer 210, together with the blowers 209 provided for the hood 106, and are controlled by the control computer 210. In other words, the gene examination apparatus 100 further includes the temperature adjusting devices 211 which drive the plate-like heaters 105, and the control computer 210 which controls the temperature adjusting devices 211 and the blowers 209.

As shown in FIG. 13, the temperature adjusting mechanism preferably includes temperature sensors 212 which measure the surface temperatures of the plate-like heaters 105. The temperature sensors 212 are, for example, thermisters or thermocouples. Although not shown, they are provided for the base portion 120 and the cover portion 123. These temperature sensors are preferably provided near the plate-like heaters.

The temperature adjusting mechanism preferably includes heater control mechanisms such as thermostats which prevent the plate-like heaters 105 from being excessively heated. Such heater control mechanisms are effective in preventing the reaction vessel 101 from being undesirably excessively heated due to failures in the temperature adjusting devices 211 (see FIG. 13) which drive and control the plate-like heaters 105.

More preferably, the temperature adjusting mechanism further includes cooling means for lowering the temperature of the reaction vessel 101. The cooling means are, for example, Peltier elements (not shown) provided for the base portion 120 and the cover portion 123. The Peltier elements are preferably in surface contact with the reaction vessel 101 supported on the reaction stage 103. The Peltier elements for cooling are also electrically connected to the temperature adjusting devices 211, and are driven and controlled by the temperature adjusting devices 211. The Peltier elements may replace the plate-like heaters. Controlling a current allows the Peltier element serving as heating means in place of the heater 105 to function.

The temperature of the reaction vessel 101 is adjusted within the range of approximately 30° C. to approximately 90° C. during, for example, a hybridization reaction by the plate-like heaters 105, temperature sensors 212 (not shown), Peltier elements, and blowers 209 controlled by the control computer 210. Temperature control is determined in accordance with a reaction which is performed by the DNA microarray 110a, and includes control for keeping the temperature of the reaction vessel 101 at a predetermined temperature and control for heating or cooling between a plurality of preset temperature values in accordance with a predetermined temperature gradient.

On the preferred reaction stage 103, the plate-like heaters 105 for heating are in contact with the reaction vessel 101, and hence the temperature of the reaction vessel 101 can be raised within a short period of time.

According to the preferred gene examination apparatus 100, the examination apparatus comprises by itself the blowers 209 for cooling, and the reaction stage 103 comprises Peltier elements for cooling. In the examination apparatus 100 having such an arrangement, the temperature of the reaction vessel 101 can be lowered within a short period of time by the blowers 209 and the Peltier elements at once.

Gene examining operation will be described below.

As shown in FIG. 8, while the cover portion 123 is open, the reaction vessel 101 is placed on the base portion 120 of the reaction stage 103. The nucleic acid sample solution 114 is then supplied to each opening portion 113 of the reaction vessel 101. That is, the nucleic acid sample solution 114 is stored in the DNA microarray 110a. Thereafter, as shown in FIG. 9, the cover portion 123 is closed.

With this operation, as shown in FIG. 10, the waste liquid hole 116 of the reaction vessel 101 is fluidally communicated with the discharge channel 119 provided for the base portion 120, and the liquid storage hole 117 of the reaction vessel 101 is fluidally communicated with the transfer channel provided for the syringe piston pump unit 125. Note that all the pipings of the syringe piston pump unit 125 of the reaction stage 103 are filled with the washing water 137 in advance.

While the cover portion 123 is closed, as shown in FIG. 10, the cover glass 121 provided for the cover portion 123 is in surface contact with the upper housing 108 of the reaction vessel 101. Accordingly, the opening portions 113 (the portions through which the nucleic acid sample solution 114 is stored in the DNA microarrays 110a) formed in the upper housing 108 of the reaction vessel 101 is covered.

This arrangement prevents foreign substances such as dust in the hood 106 from entering the opening portions 113 of the upper housing 108 of the reaction vessel 101. This makes it possible to prevent the occurrence of an examination error in the DNA microarray 110a with the microscope 102 due to the mixing of a foreign substance.

Thereafter, the temperature of the DNA microarray 110a in the reaction vessel 101 is controlled to cause the nucleic acid sample to react.

Temperature control on the DNA microarray 110a will be described below.

Referring to FIG. 13, predetermined heating temperature information 213 of the DNA microarray 110a is input to the control computer 210, which in turn outputs a signal representing the predetermined input heating temperature information 213 to the temperature adjusting device 211. The temperature adjusting device 211 receives a signal indicating the surface temperature of the heater 105 which is output from the temperature sensor 212. If the received surface temperature information of the heater 105 has not reached a predetermined range of a predetermined temperature 213 input to the control computer 210, the temperature adjusting device 211 outputs a signal for heating to the heater 105. This operation is repeated until the temperature of the heater is raised to a predetermined temperature on the basis of a predetermined temperature gradient which has been set in the temperature adjusting device 211.

On the other hand, if the surface temperature information of the heater 105 which the temperature adjusting device 211 has received from the temperature sensor 212 exceeds the predetermined range of predetermined temperature information 213 which has been input to the control computer 210, the temperature adjusting device 211 outputs a signal for stopping to the heater 105. In addition, the control computer 210 may output a signal for rotating the blower 209, as needed, to shorten the cooling time until the surface temperature information of the heater 105 becomes a predetermined temperature or less.

The DNA microarrays 110a and the respective liquids are heated to predetermined temperatures by the four heaters 105.

Since each opening portion 113 of the reaction vessel 101 is covered by the cover glass 121, a high-humidity environment is set inside the opening portion. For this reason, the nucleic acid sample solution 114 does not evaporate, and the concentration of the nucleic acid sample solution 114 is kept constant. Therefore, no undesired change occurs in the concentration of the nucleic acid sample solution 114.

Since each opening portions 113 of the reaction vessel 101 is blocked from an external environment by the two cover glasses 121 which are placed through a sealed air space, a temperature gradient with respect to the outer environment is reduced. This prevents dew condensation on the surfaces of the cover glasses 121 during temperature control.

Subsequently, only the electromagnetic valve 127 on the DNA microarray 110a side is opened, and the piston 129 of the syringe piston pump 131 is moved to the right on the drawing surface toward the suction side, as shown in FIG. 11. Consequently, the nucleic acid sample solution 114 passes through the DNA microarray 110a and moves into the liquid storage hole 117 in the lower housing 109. Subsequently, as shown in FIG. 10, the piston 129 is moved to the left on the drawing surface toward the discharge side. Consequently, the nucleic acid sample solution 114 passes through the DNA microarray 110a, and returns to the upper side of the DNA microarray 110a, i.e., the interior of the opening portions 113. This operation is repeatedly performed. With this operation, the nucleic acid sample causes a hybridization reaction with the nucleic acid probe spot 118 in the DNA microarray 110a. As a result, only the nucleic acid probe spot 118 can emit fluorescence.

As shown in FIG. 11, thereafter, while all the amount of nucleic acid sample solution 114 is stored in the liquid storage hole 117, fluorescence observation is performed with the microscope 102 to examine weak fluorescence from each hybridized nucleic acid probe spot 118. At this time, the nucleic acid sample solution 114 is removed from the tapered opening portions 113 of the upper housing 108 above the DNA microarray 110a.

The volume of the liquid storage hole 117 is determined in advance to inhibit the nucleic acid sample solution 114 from reaching the interior of the syringe piston pump unit 125, and the syringe piston pump 131 is driven such that the moving distance of the piston 129 is limited in accordance with the volume of the liquid storage hole 117. This prevents the nucleic acid sample solution 114 from entering the syringe piston pump unit 125, and hence no carry over occurs when another DNA microarray 110a is set.

After all items concerning the DNA microarrays 110a placed on the reaction stage 103 are complete, the control computer 210 outputs rotation signals to the blowers 209. The blowers 209 feed air to the reaction stage 103. The temperature sensors 212 send signals representing the surface temperatures of the heaters 105 to the control computer 210 through the temperature adjusting devices 211. When the temperature represented by each signal becomes a predetermined temperature (a temperature at which the user suffers no burn even if he/she touches the stage 103) or less which is input in advance, the control computer 210 draws out the reaction stage 103 from the hood 106. However, the step of causing the blowers 209 to feed air is not always required. Cooling can be performed by natural cooling, circulating cooling water, or using a Peltier element. Therefore, when the temperature of the reaction stage becomes the predetermined temperature or less, the stage is drawn out from the hood, allowing the apparatus to be safely operated.

The condition under which the reaction stage 103 is drawn out from the hood 106 is not necessarily limited to that examination is complete with respect to all the items concerning the DNA microarrays 110a. For example, the reaction stage 103 may be drawn out between the respective examination items to allow the user to dispense a new nucleic acid sample solution 114, cleaning fluid, or another liquid into the tapered opening portion 113 of the upper housing 108 of the DNA microarray 110a as needed.

At this time, the reaction stage 103 does not necessarily require to be cooled to a predetermined temperature or less by the blowers 209. If, for example, a predetermined temperature at the next examination item is higher than a predetermined temperature for hybridization at the previous examination item, the stage 103 may be drawn out from the hood 106 without being cooled. In this case, information indicating that the temperature of the stage is high is preferably displayed on the stage, the monitor, or the like.

The nucleic acid sample solution 114 may be removed from the DNA microarray 110a by the suction pump 135 instead of being transferred to the liquid storage hole 117. In this case as well, the S/N ratio of the fluorescence intensity at each spot increases.

More preferably, however, the DNA microarray 110a is cleaned by cleaning fluid after the end of a hybridization reaction. After a hybridization reaction is performed by causing the nucleic acid sample solution 114 to pass through the DNA microarray 110a, the washing water 137 is supplied onto the DNA microarray 110a by the syringe piston pump 131. Thereafter, the electromagnetic valve 128 is opened, and the electromagnetic valve 127 is closed. The suction pump 135 is then operated to discharge the nucleic acid sample solution 114, the washing water 137, and the like from the reaction vessel 101. Accordingly, not only is the residual nucleic acid sample solution 114 removed but also the DNA microarray 110a is cleaned with the washing water 137.

Such cleaning operation and waste liquid sucking operation may be repeatedly performed, so that the residual nucleic acid sample solution 114 is reliably removed.

In the gene examination apparatus 100 of this embodiment, the reaction stage 103 mounted on the X-Y stage of the microscope 102 comprises the heaters 105 and the syringe piston pumps 131, and hence there is no need to transfer the DNA microarrays 110a in the reaction vessel 101 between the respective devices. This shortens the time required for transfer, and hence examination can be performed at high speed. This can eliminate changes in temperature of each DNA microarray, which has occurred at the time of transfer. Since this can eliminate the adhesion of dust to each DNA microarray, which has occurred at the time of transfer, highly accurate examination can be performed. Since there is no need to perform transfer, examination is facilitated.

This embodiment has exemplified DNA microarrays. Since a DNA microarray needs to detect a weak signal with high accuracy, the present invention is very effective in improving the accuracy. However, the present invention can be applied to any technique as long as it is designed to perform detection by immobilizing a probe on a porous solid support. Biorelated substances which can be used in this case include not only cells of animals, plants, microorganisms, and the like but also substances originating from viruses which cannot proliferate by themselves unless parasitizing such cells. Biorelated substances include substances in natural forms which are directly extracted or isolated from these cells, substances produced by using a gene optical technique, and chemically modified substances. More specifically, biorelated substances include hormones, enzymes, antibodies, antigens, abzymes, other proteins, nucleic acids, and the like.

In addition, a probe means a substance which specifically binds with the above biorelated substance, and includes any one of substances in the following relationships: a ligand such as a hormone and its acceptor, an enzyme and its substrate, an antigen and its antibody, a nucleic acid having a specific sequence and a nucleic acid having a sequence complementary thereto, and the like.

The gene examination apparatus 100 of this embodiment may be variously changed and modified.

FIG. 14 shows a modification of the syringe piston pump which can replace the syringe piston pump 131 shown in FIGS. 10 and 11. As shown in FIG. 14, a syringe piston pump 205 of this modification may be configured such that a groove 208 is formed in the distal end portion of a piston 207, on the liquid transfer/suction port side (on the left side with respect to the drawing surface), which can move in a syringe 206, an O-ring is fitted in the groove 208, and the O-ring 139 slides on the inner wall of the syringe 206 together with the movement of the piston 207.

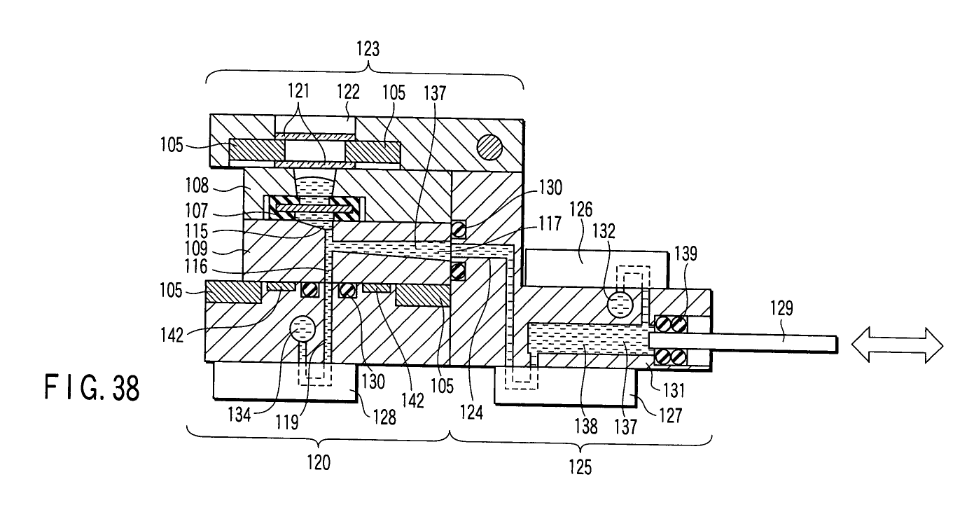

FIG. 38 shows a modification of the reaction stage shown in FIGS. 10 and 11. The reaction stage of this modification has an arrangement in which Peltier elements 142 for lowering the temperature of the reaction vessel 101 are added to the reaction stage shown in FIGS. 10 and 11.

As shown in FIG. 38, in the reaction stage of this modification, the base portion 120 further includes the Peltier elements 142 for lowering the temperature of the reaction vessel 101. The Peltier elements 142 are in surface contact with the reaction vessel 101 supported on the reaction stage 103. The Peltier elements 142 are electrically connected to the temperature adjusting devices 211, and are driven/controlled by the temperature adjusting devices 211.

In this reaction stage, the reaction vessel 101 is heated by the heaters 105 and is cooled by the Peltier elements 142.

### Second Embodiment

This embodiment is directed to a base portion which has piezoelectric elements in order to improve the cleaning effect for DNA microarrays with washing water, which has been described in the first embodiment.

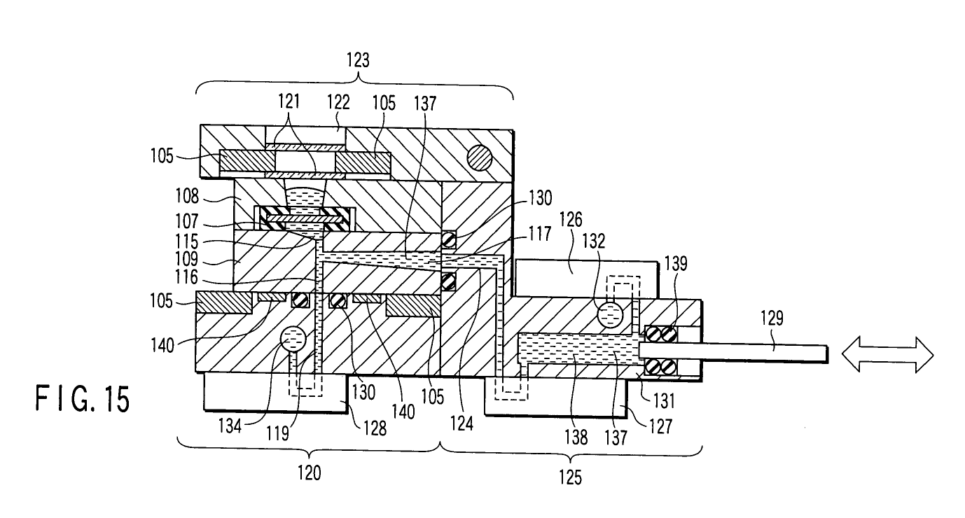

As shown in FIG. 15, a base portion 120 comprises two piezoelectric elements 140 which are in surface contact with a lower housing 109 of a reaction vessel 101 mounted on the base portion 120. Each piezoelectric element 140 has a size of, for example, 10 mm×30 mm×1 mm. The piezoelectric element 140 comprises a lead titanate zirconate (PZT) plate having undergone polarization processing in the thickness direction and silver electrodes deposited on the two surfaces in the thickness direction.

A sine wave alternating voltage of approximately 100 Vp-p is applied from a driving circuit (not shown) to the piezoelectric element 140 using a flexible cable or the like. When an alternating voltage having such a frequency is applied, the piezoelectric element 140 generates strong ultrasonic vibration.

As shown in FIG. 15, after the end of a hybridization reaction, washing water 137 is transferred onto each DNA microarray 110a by a syringe piston pump 131. While the washing water 137 is supplied onto the DNA microarray 110a in this manner, the piezoelectric elements 140 incorporated in the base portion 120 are driven by sine wave alternating voltages. The piezoelectric elements 140 then generate ultrasonic vibration. With this operation, the portion to which the washing water is supplied is cleaned by ultrasonic cleaning.

Subsequently, a waste liquid electromagnetic valves 128 is opened, an electromagnetic valve 127 is closed, and a suction pump 135 is operated, so that a residual nucleic acid sample solution 114 and the washing water 137 are removed from the reaction vessel 101.

At this time, the cleaning effect can be improved by applying ultrasonic vibration when cleaning is performed with washing water. As a result, the nucleic acid sample exists in only proper nucleic acid probe spots which have caused a hybridization reaction, and the residual nucleic acid sample hardly exists. This further reduces undesired fluorescence and further increases the S/N ratio of fluorescence observation.

### Third Embodiment

This embodiment is directed to an examination apparatus having a function of supplying washing water to DNA microarrays from above or a function of sucking washing water from above the DNA microarrays at the time of cleaning of the DNA microarrays described in the first embodiment.

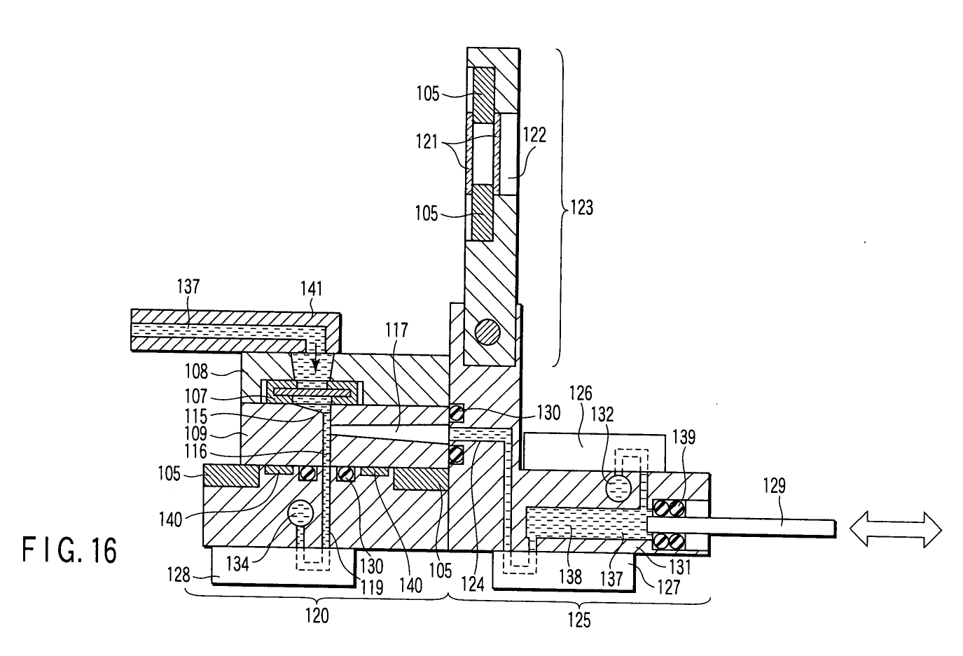

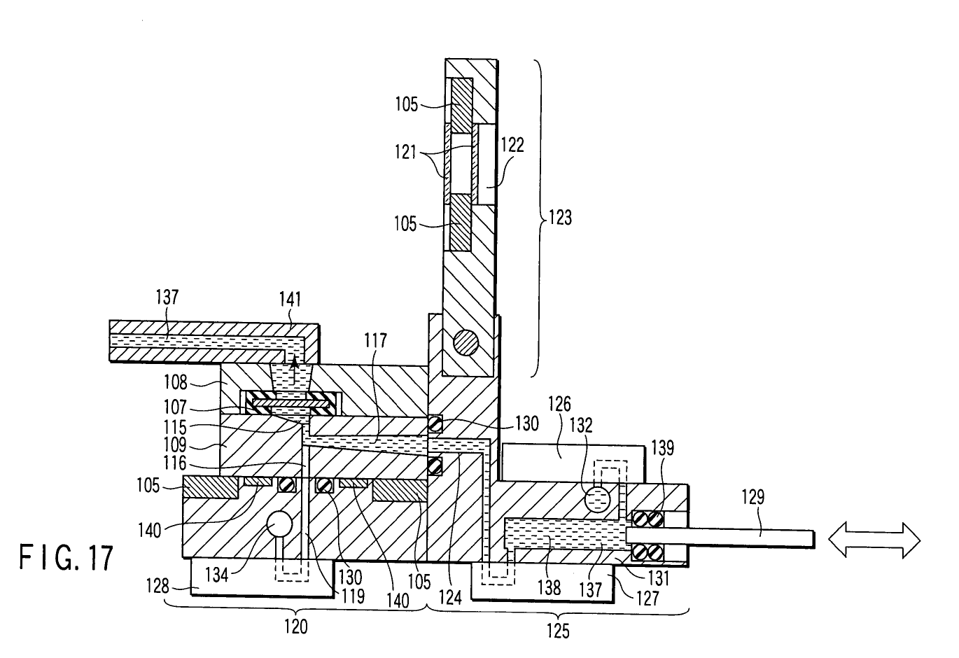

As shown in FIGS. 16 and 17, the examination apparatus further includes cleaning nozzles 141 which supply washing water to DNA microarrays from above, i.e., from an examination surface side. Each cleaning nozzle 141 is brought into contact with an upper housing 108 of a reaction vessel 101 and supplies washing water to a tapered opening portions 113 formed in the upper housing 108 while a cover portion 123 of a reaction stage 103 is open. Although not shown, the cleaning nozzle 141 is supported by another transfer member, and a pump which transfers washing water 137 to a washing water tank 133 is connected to the cleaning nozzle 141.

After the end of a hybridization reaction, as shown in FIG. 16, the cover portion 123 is opened, and the cleaning nozzle 141 is brought into contact with the upper housing 108 of the reaction vessel 101 to supply washing fluid to the tapered opening portions 113 formed in the upper housing 108. Note, however, that the cleaning nozzle 141 need not always come into contact with the upper housing 108, and can supply washing fluid without contact.

While washing fluid is supplied from the cleaning nozzle 141, only a waste liquid electromagnetic valve 128 is opened, and a suction pump 135 is operated. With this operation, the washing water 137 is discharged from the reaction vessel 101, together with a residual nucleic acid sample.

As in the second embodiment, a base portion 120 comprises piezoelectric elements 140 which improve the cleaning effect, and ultrasonic cleaning with the piezoelectric elements 140 may also be used during this cleaning operation.

As shown in FIG. 17, while the washing water 137 in the washing water tank 133 is transferred to a DNA microarray 110a by a syringe piston pump 131, the washing water is sucked and removed by the cleaning nozzle 141. With this operation, a residual nucleic acid sample is removed.

In this embodiment, since washing water flows in one direction during cleaning operation, the DNA microarray 110a is always cleaned with clean washing water. This greatly shortens the cleaning time. In addition, since washing water is sucked and removed by the cleaning nozzle located above the DNA microarray 110a, dust existing above the DNA microarray 110a, i.e., on the examination surface side, is removed together.

As a result, the nucleic acid sample exists in only a proper nucleic acid probe spot which has caused a hybridization reaction, and the extra residual nucleic acid sample hardly exists. This further reduces undesired fluorescence and further increases the S/N ratio of fluorescence observation.

A substance for cleaning DNA microarrays is not limited to a liquid. That is, a substance to be supplied from the cleaning nozzles 141 may be temperature-controlled fine liquid droplets. Temperature-controlled fine liquid droplets can also remove the residual nucleic acid sample with high efficiency. In order to suppress a pressure load when steam liquid droplets pass through DNA microarrays, the size of each liquid droplet is preferably equal to or less than several μm or less. A substance to be supplied from the cleaning nozzles 141 may be air. In this case, the opening portions 113 of the reaction vessel 101 need to be sealed with the cleaning nozzles 141 or another means.

### Fourth Embodiment

This embodiment is directed to a reaction stage which transfers a nucleic acid sample solution by air. This embodiment is directed to an examination apparatus in which the syringe piston pump unit in the first embodiment is changed into an arrangement for transferring a fluid by air.

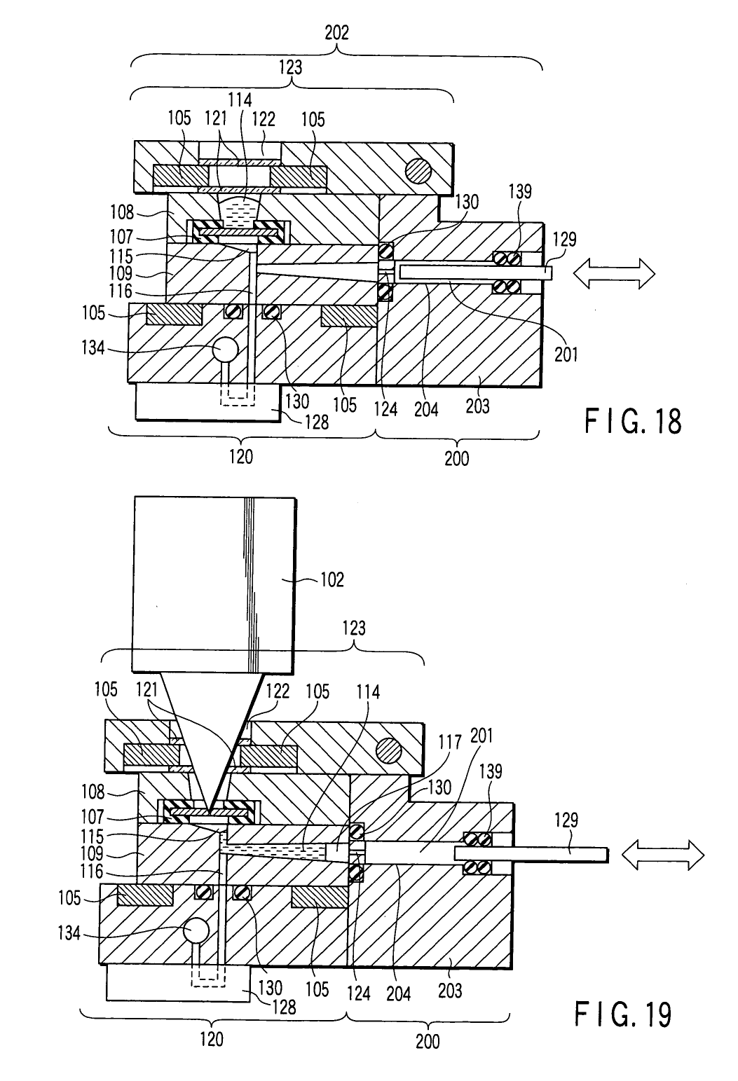

As shown in FIGS. 18 and 19, a reaction stage 202 in this embodiment includes a syringe piston pump unit 200 which is a fluid transfer mechanism for transferring a fluid with respect to a reaction vessel 101.

The syringe piston pump unit 200 includes four transfer channels 124 which are fluidally communicated with liquid storage holes 117 of the reaction vessel 101, respectively, and four syringe piston pumps 203 which are fluidally communicated with the liquid storage holes 117 of the reaction vessel 101, respectively.

Each syringe piston pump 203 includes a syringe 204, a piston 129 which can move in the syringe 204, and a seal 139 which is a seal for keeping the syringe 204 and the piston 129 in an airtight state.

A nucleic acid sample solution in the reaction vessel 101 supported on a reaction stage 202 is transferred by the suction and discharge of air 201 upon movement of the piston 129.

By selectively driving four electromagnetic valves 128, an arbitrary one of four DNA microarrays 110a can be selectively used for examination.

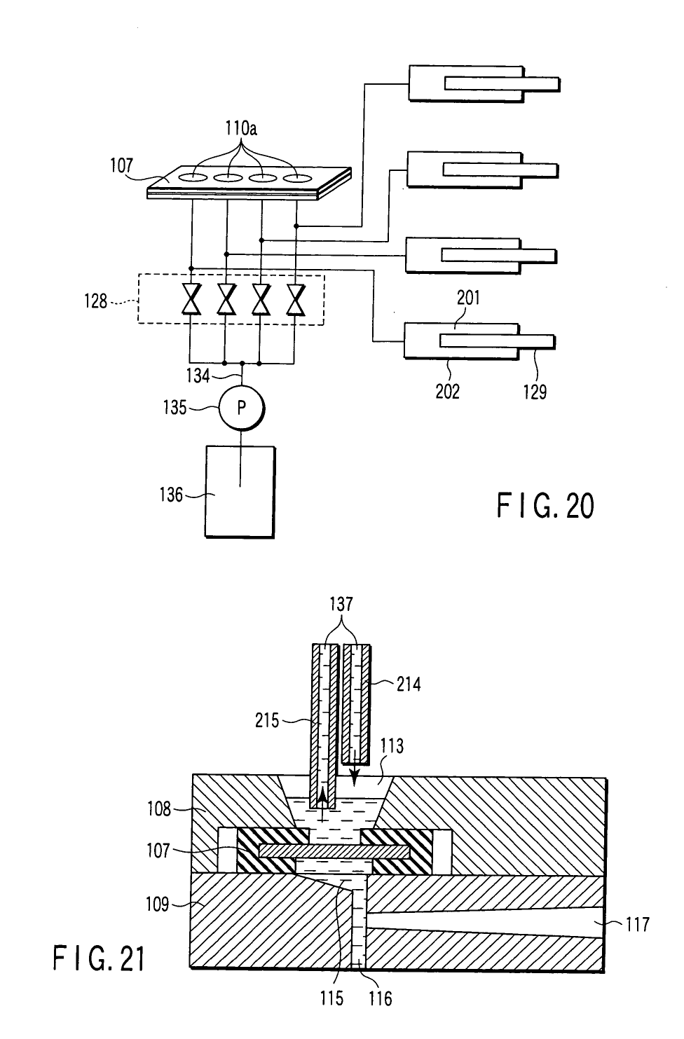

A base portion 120 is similar to that in the first embodiment. As schematically shown in FIG. 20, a waste liquid collecting pipe 134 of the base portion 120 is in communication with a waste liquid tank 136 through a suction pump 135.

A cover portion 123 is opened, and the reaction vessel 101 is placed on the base portion 120 of a reaction stage 103. A nucleic acid sample solution 114 is supplied in each opening portions 113 of the reaction vessel 101. Thereafter, as shown in FIG. 18, the cover portion 123 is closed.

With this operation, the liquid storage hole 117 of the reaction vessel 101 is fluidally communicated with the syringe piston pump 203 through only the transfer channel 124. In addition, a waste liquid hole 116 of the reaction vessel 101 is fluidally communicated with a discharge channel 119 provided for the base portion 120.

Subsequently, only the electromagnetic valve 128 on the DNA microarray 110a side is closed, and the piston 129 of the syringe piston pump 203 is moved to the right on the drawing surface toward the suction side, as shown in FIG. 19. With this operation, since air in the reaction vessel 101 is sucked into the syringe piston pump 203, the nucleic acid sample solution 114 passes through the DNA microarray 110a and moves into the liquid storage hole 117 in the lower housing 109. As shown in FIG. 18, the piston 129 is then moved to the left on the drawing surface toward the discharge side. With this operation, since air in the syringe piston pump 203 is discharged into the reaction vessel 101, the nucleic acid sample solution 114 passes through the DNA microarray 110a and returns into the opening portion 113. When this operation is repeatedly performed, the nucleic acid sample causes a hybridization reaction with the nucleic acid probe spot 118 in the DNA microarray 110a within a short period of time. As a result, only the nucleic acid probe spot 118 can emit fluorescence.

After the hybridization reaction, the DNA microarray 110a is cleaned by the same technique as that in the third embodiment. That is, a cleaning nozzle 141 is set for the reaction vessel 101 from the examination surface side, and washing water 137 is supplied from the cleaning nozzle 141. In addition, the supplied washing water 137 is removed by the suction pump 135 through the waste liquid hole 116.

In this embodiment, the transfer channel 124 which communicates between the syringe piston pump 203 and the reaction vessel 101 is not provided with any electromagnetic valve midway along the channel, and hence is short and has no bent portion. For this reason, the movement responsiveness of the nucleic acid sample solution 114 and washing water 137 based on the syringe piston pump 203 is good.

The syringe piston pump unit 200 has no electromagnetic valve, and hence is reduced in size and power consumption and is provided at a low cost.

Using the gas (air) 201 in place of the washing water 137 in the piping of the syringe piston pump unit 200 eliminates the possibility that when the user removes the reaction vessel 101 from the base portion 120, the liquid storage hole 117 touches the atmosphere to generate a negative pressure in the liquid storage hole 117, which causes the washing water 137 in the liquid transfer port which has been in contact with the liquid storage hole 117 to leak from the syringe piston pump unit 200, decreasing the amount of washing water 137, so that the movement responsiveness of the nucleic acid sample solution 114 which passes through the porous portion 110 and the liquid storage hole 117 decreases.

In addition, using the gas (air) 201 in place of washing water in the piping of the syringe piston pump unit 200 eliminates the labor of filling the piping with the washing water 137 and the time to wait for filling.

Furthermore, using the gas (air) 201 in place of washing water in the piping of the syringe piston pump unit 200 eliminates labor in maintenance, e.g., removing all the washing water 137 before an inspection of the apparatus, thereby improving the maintainability.

In the embodiment described above, when the piping of the syringe piston pump unit 200 is filled with the washing water 137, air may mix with the water to form an air layer. This air layer may degrade the movement responsiveness of the nucleic acid sample solution 114 which is made to pass through the porous portion 110 and the liquid storage hole 117 by the syringe piston pump 203. In this embodiment, however, since the piping is filled with the gas (air) 201, such a factor, i.e., an air layer, which degrades the movement responsiveness can be eliminated.

In this embodiment as well, although not shown in particular, the syringe piston pump unit 200 may include a pressure sensor for detecting the pressure of the air 201 to be sucked and discharged.

### Fifth Embodiment

This embodiment is directed to a gene examination apparatus comprising a cleaning nozzle which supplies washing water and a waste liquid nozzle which removes washing water.

As shown in FIG. 21, the gene examination apparatus according to this embodiment includes a cleaning nozzle 214 which supplies washing water 137 to a tapered opening portion 113 formed in an upper housing 108 of a reaction vessel 101 from above, and a waste liquid nozzle 215 which removes the washing water 137. The cleaning nozzle 214 includes a liquid transfer mechanism comprising a rod having, for example, a pressure means, and a nozzle chip fitted in the rod.

The cleaning nozzle 214 is connected to a pump (not shown) for transferring the washing water 137 in a washing water tank 133. The waste liquid nozzle 215 is connected to a pump (not shown) for discharging the washing water 137 from the tapered opening portion 113 of the upper housing 108 to a waste liquid tank 136. The end portion, i.e., the suction portion, of the waste liquid nozzle 215 is preferably placed near a DNA microarray. With this arrangement, the remaining amount of liquid at the examination surface of the washing water 137 decreases. In addition, the cleaning nozzle 214 is preferably located at a position where it does not come into contact with the washing water 137 stored in the tapered opening portion 113 of the upper housing 108. This prevents contamination with a nucleic acid sample.

Although not shown in FIG. 21, the reaction vessel 101 is supported on a reaction stage 103 described in the first embodiment. That is, a liquid storage hole 117 of the reaction vessel 101 is communicated with a syringe piston pump 131.

For example, the washing water 137 is supplied from the cleaning nozzle 214 into the tapered opening portion 113 of the upper housing 108. With the sucking operation of the syringe piston pump 131, the washing water 137 in the opening portion 113 passes through the DNA microarray and moves into the liquid storage hole 117. Thereafter, with the discharging operation of the syringe piston pump 131, the washing water 137 in the liquid storage hole 117 passes through the DNA microarray and moves into the opening portion 113.

After the sucking operation and discharging operation of the syringe piston pump 131 are properly repeated, the washing water 137 in the opening portion 113 is sucked by the waste liquid nozzle 215 and removed. This removes foreign substances existing on the DNA microarray, together with the washing water 137.

If the washing water 137 left on the DNA microarray influences an examination result after suction by the waste liquid nozzle 215, the washing water 137 is preferably discharged by a suction pump 135 through a waste liquid hole 116.

The washing water 137 may be supplied from the syringe piston pump 131 as well as from the cleaning nozzle 214.

For example, after a hybridization reaction, the washing water 137 is supplied onto the DNA microarray by the syringe piston pump 131. The washing water 137 is then sucked by the waste liquid nozzle 215. With this operation, foreign substances existing on the DNA microarray are removed, together with the washing water 137. Subsequently, the washing water 137 is supplied from the cleaning nozzle 214, and is sucked by the suction pump 135. With this operation, the washing water 137 passes through the DNA microarray and is removed from the reaction vessel 101 through the waste liquid hole 116.

The above sequence of supply of the washing water 137 may be reversed. That is, after the washing water 137 is supplied from the cleaning nozzle 214 and is discharged from the reaction vessel 101 by the suction pump 135, the washing water 137 may be supplied onto a DNA microarray by the syringe piston pump 131, and the washing water 137 on the DNA microarray may be sucked by the waste liquid nozzle 215.

In this embodiment, since the washing water 137 is sucked by the waste liquid nozzle 215 placed above each DNA microarray, foreign substances existing on the DNA microarray are removed, together with the washing water.

Performing both discharge of the washing water 137 by the suction pump 135 and discharge of the washing water 137 by the waste liquid nozzle 215 enables the discharge time for the washing water 137 to be shortened.

Performing both supply of the washing water 137 by the syringe piston pump 131 and supply of the washing water 137 by the cleaning nozzle 214 enables the supply time for the washing water 137 to be shortened.

### Sixth Embodiment

This embodiment is directed to a reaction vessel 101 including a fluid transfer mechanism.

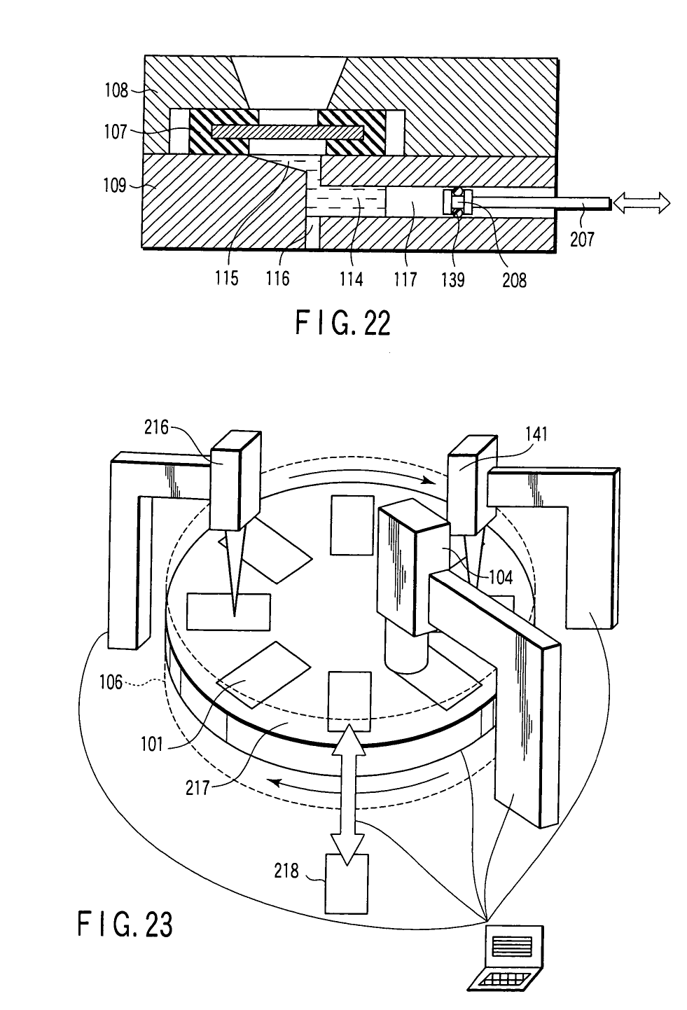

As shown in FIG. 22, a liquid storage hole 117 of a lower housing 109 of the reaction vessel 101 of this embodiment has a circular column shape or polygonal column shape, and also serves as the syringe of a syringe piston pump. A piston 207 is provided so as to be movable in the liquid storage hole 117. The piston 207 has a groove 208 formed in its end portion. A seal 139 is fitted in the groove 208. The seal 139 keeps the piston 207 and the liquid storage hole 117, i.e., the syringe, in an airtight state.

A reaction stage which supports the reaction vessel 101 of this embodiment may have an arrangement in which the syringe piston pump unit 125 is omitted from the reaction stage 103 in the first embodiment. The piston 207 is inserted into the liquid storage hole 117 of the reaction vessel 101 before the reaction vessel 101 is placed on the reaction stage. After the reaction vessel 101 is placed on the reaction stage, the piston 207 is coupled to a motor unit (not shown), and can be moved to left and right in FIG. 22 by the motor unit.

After a nucleic acid sample solution 114 is dispensed, the nucleic acid sample solution 114 may or may not come into contact with the piston 207.

In this embodiment, since the syringe piston pump is provided for the reaction vessel 101, the air layer between the nucleic acid sample solution 114 and the piston 207 is small. This improves the movement responsiveness of the nucleic acid sample solution 114.

In addition, since the reaction vessel 101 in this embodiment includes the syringe piston pump, the reaction stage which supports the reaction vessel can be reduced in size. Therefore, the gene examination apparatus can be reduced in size.

### Seventh Embodiment

This embodiment is directed to a gene examination apparatus including a circular reaction stage which can support a plurality of reaction vessels 101.

As shown in FIG. 23, the gene examination apparatus according to this embodiment includes a circular reaction stage 217 which is controlled as a whole by a control computer 210 as described above and can be rotated about the center of the stage as an axis, a hood 106 which covers the stage, and a CCD camera 104 for fluorescence observation of the DNA microarrays in the reaction vessels 101 supported and rotated by the circular reaction stage 217.

The circular reaction stage 217 can radially support the plurality of reaction vessels 101. That is, the circular reaction stage 217 is provided with a plurality of support mechanisms for supporting the reaction vessels 101. Although not shown, the circular reaction stage 217 includes a fluid transfer mechanism for transferring a fluid with respect to the supported reaction vessels 101 and a temperature adjusting mechanism for adjusting the temperatures of DNA microarrays in the supported reaction vessels 101. The arrangements in the above embodiments can be applied to the support mechanism, fluid transfer mechanism, and temperature adjusting mechanism.

The gene examination apparatus preferably includes a nucleic acid sample solution dispensing nozzle 216 for dispensing a nucleic acid sample solution into the reaction vessel 101 which is supported and rotated by the circular reaction stage 217. More preferably, the gene examination apparatus further includes a cleaning nozzle 141 for cleaning the DNA microarray in the reaction vessel 101 which is supported and rotated by the circular reaction stage 217. The nucleic acid sample solution dispensing nozzle 216, cleaning nozzle 141, and CCD camera 104 are arranged around the circular reaction stage 217 along its rotating direction.

For example, when the reaction vessel 101 is to be placed on the circular reaction stage 217, the support mechanism is drawn out from the hood 106 by a Y stage (not shown) to facilitate placement. After the reaction vessel 101 is placed, the support mechanism is housed in the hood 106 again by the Y stage.

The reaction vessel 101 supported on the circular reaction stage 217 is transferred to the work area of the nucleic acid sample solution dispensing nozzle 216 by the rotation of the circular reaction stage 217. In this area, a nucleic acid sample solution is dispensed into the reaction vessel 101 by the nucleic acid sample solution dispensing nozzle 216.

While the reaction vessel 101 supported by the circular reaction stage 217 is transferred by the rotation of the circular reaction stage 217 after the nucleic acid sample solution is dispensed, a hybridization reaction is performed. After the hybridization reaction, the reaction vessel 101 supported by the circular reaction stage 217 is transferred to the work area of the cleaning nozzle 141 by the rotation of the circular reaction stage 217. In this area, the DNA microarrays in the reaction vessel 101 are cleaned. FIG. 23 exemplifies the case wherein one cleaning nozzle is used. However, a plurality of nozzles for the supply of washing water and the suction of washing water can be used.

After the cleaning operation, the reaction vessel 101 supported by the circular reaction stage 217 is transferred to the imaging area of the CCD camera 104 by the rotation of the circular reaction stage 217. In this area, fluorescence images of the DNA microarrays in the reaction vessel 101 are sensed. The reaction vessel 101 having undergone imaging operation is changed with a new reaction vessel 101 by a reaction vessel change means 218.

The gene examination apparatus of this embodiment can continuously examine a plurality of DNA microarrays. Although DNA microarrays have been described above, the present invention may be applied to the analysis of other biorelated substances, e.g., the analysis of the sequences, immobilized quantities, and structures of RNAs, antigen antibodies, proteins, peptides, and the like.

### Eighth Embodiment

This embodiment is directed to an examination apparatus including a reaction vessel obtained by omitting the waste liquid holes from the reaction vessel in the first embodiment and a reaction stage suitably corresponding the reaction vessel.

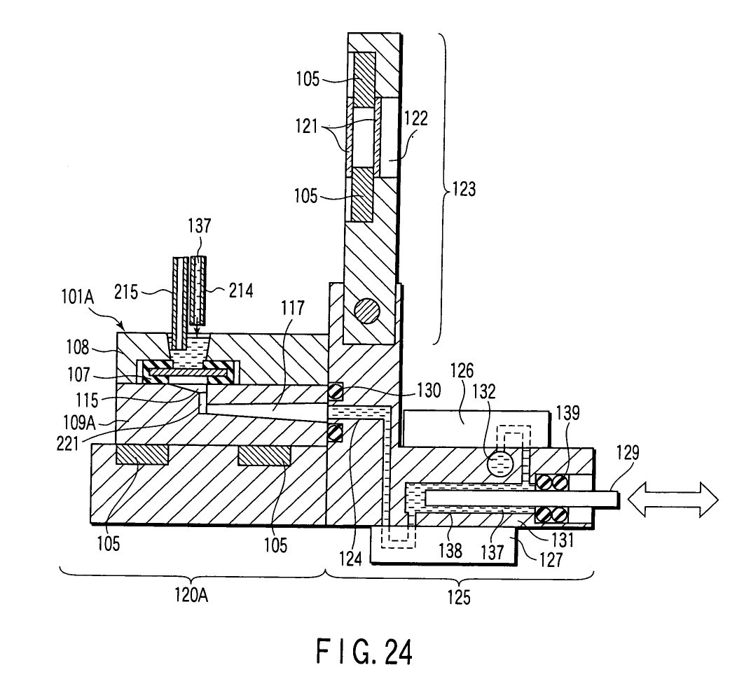

As shown in FIGS. 24 to 27, a reaction vessel 101A in this embodiment has no waste liquid hole for waste liquid which extends from the lowermost portion of a recess portion 115 to the lower surface of a lower housing 109A.

That is, the lower housing 109A includes four recess portions 115 which are formed at positions corresponding to four DNA microarrays 110a of a slide chip 107 and have inclined bottom surfaces, vertical hole portions 221 extending downward from the lowermost portions of the recess portions 115 to some midway points, and liquid storage holes 117 which extend from the lower ends of the vertical hole portions 221 to a side surface of the lower housing 109A and which can store fluids containing the nucleic acid sample to be supplied to the DNA microarrays 110a.

In correspondence with the fact that the reaction vessel 101A has no waste liquid hole, a base portion 120A in this embodiment has an arrangement obtained by omitting the discharge channels 119, the electromagnetic valves 128, and the waste liquid collecting pipe 134 from the base portion 120A in the first embodiment, and further omitting the circular grooves surrounding the terminations, i.e., the liquid transfer ports, of the discharge channels 119, and the O-rings 130 fitted in the grooves from the base portion 120A.

That is, the base portion 120A simply includes plate-like heaters 105 for heating the reaction vessel 101A. As in the second embodiment, the base portion 120A may include piezoelectric elements for performing ultrasonic cleaning to improve the cleaning effect in cleaning operation.

As in the fifth embodiment, the gene examination apparatus of this embodiment further includes cleaning nozzles 214 which supply washing water 137 to the reaction vessel 101A from above and waste liquid nozzles 215 which remove the washing water 137 from the reaction vessel 101A.

As has already been described in the fifth embodiment, each cleaning nozzle 214 is connected to a pump (not shown) for transferring the washing water 137 in a washing water tank 133. Each waste liquid nozzle 215 is connected to a pump (not shown) for discharging the washing water 137 from a tapered opening portion 113 of an upper housing 108 to a waste liquid tank 136.

The end portion, i.e., the suction port, of the waste liquid nozzle 215 is preferably located near a DNA microarray. With this arrangement, the remaining amount of liquid on the examination surface of the washing water 137 decreases. In addition, the cleaning nozzle 214 is preferably located at a position where it does not come into contact with the washing water 137 stored in the tapered opening portion 113 of the upper housing 108. This prevents contamination with a nucleic acid sample.

Other arrangements are the same as those of the first embodiment.

As in the first embodiment, the reaction vessel 101A is supported on a reaction stage 103, and the liquid storage hole 117 is fluidally communicated with a syringe piston pump 131. A hybridization reaction is performed in the same manner as in the first embodiment.

After the hybridization reaction, as shown in FIG. 24, for example, the washing water 137 is supplied from the cleaning nozzle 214 into the tapered opening portion 113 of the upper housing 108. With the sucking operation of the syringe piston pump 131, the washing water 137 in the tapered opening portion 113 passes through the DNA microarray and moves into the liquid storage hole 117. Thereafter, with the discharging operation of the syringe piston pump 131, the washing water 137 in the liquid storage hole 117 passes through the DNA microarray and moves into the opening portion 113.

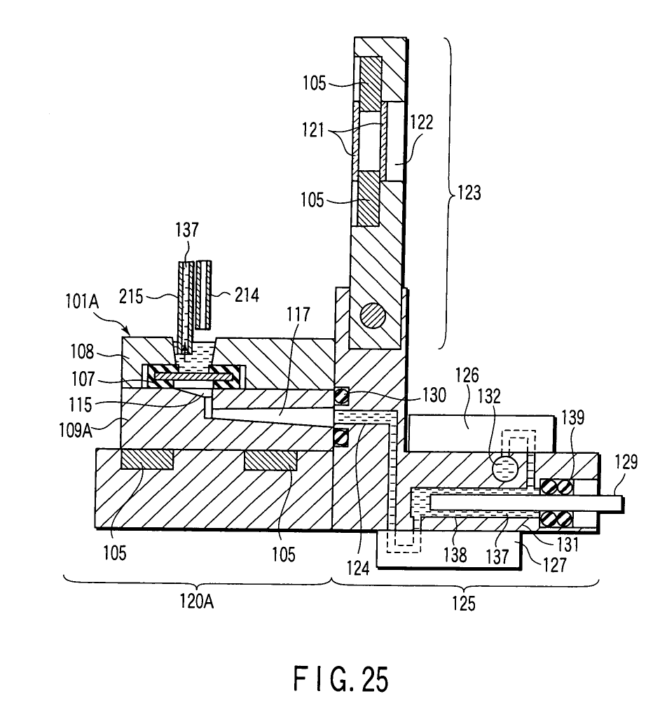

After the sucking operation and discharging operation of the syringe piston pump 131 are properly repeated, the washing water 137 in the opening portion 113 is sucked by the waste liquid nozzle 215 and removed, as shown in FIG. 25. This removes foreign substances existing on the DNA microarray, together with the washing water 137.

If the washing water 137 left on the DNA microarray influences an examination result after suction by the waste liquid nozzle 215, the washing water 137 is preferably sucked into the liquid storage hole 117 by the suction pump 131.

Although FIGS. 24 and 25 have exemplified the case wherein the washing water 137 is supplied from the cleaning nozzle 214, the washing water 137 may also be supplied from the syringe piston pump 131.

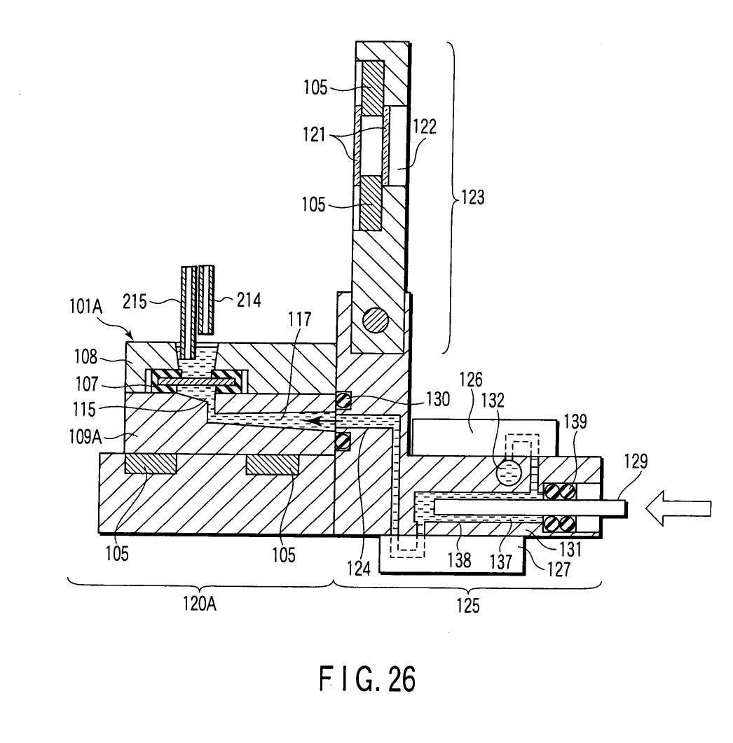

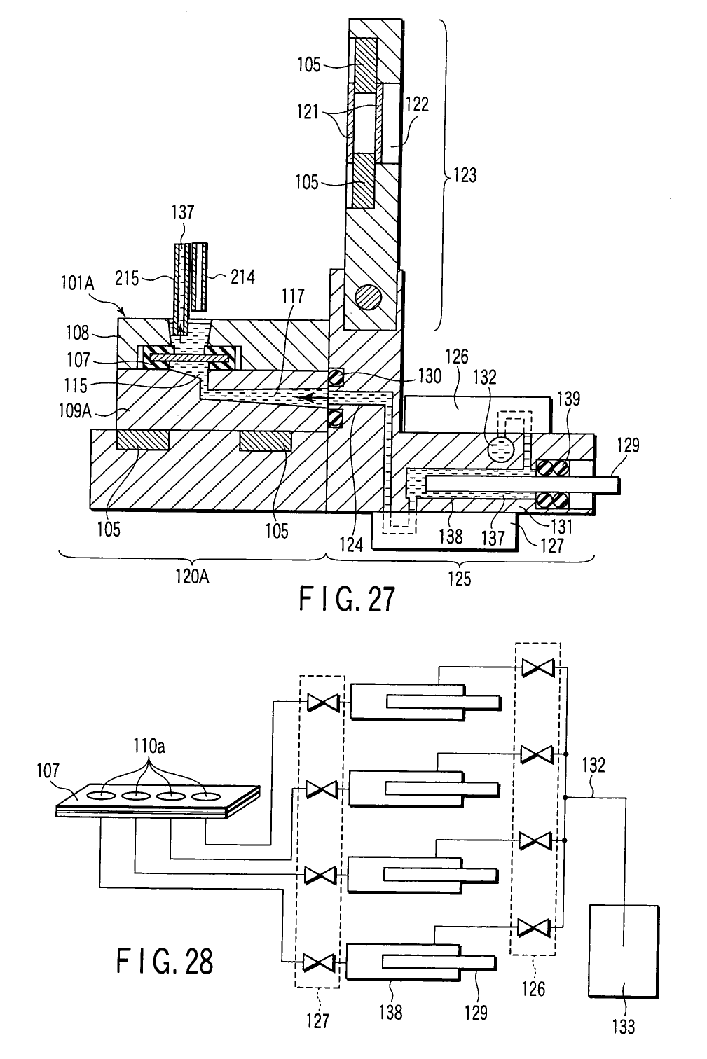

For example, after a hybridization reaction, the washing water 137 is supplied onto the DNA microarray by the syringe piston pump 131, as shown in FIG. 26. The washing water 137 is then sucked by the waste liquid nozzle 215, as shown in FIG. 27.

The supply of the washing water 137 by the syringe piston pump 131 and the suction of the washing water 137 by the cleaning nozzle 214 may be repeated by a proper number of times, as needed.

In this embodiment, since the washing water 137 is sucked by the waste liquid nozzle 215 placed above the DNA microarray, foreign substances existing on the DNA microarray are removed, together with the washing water.

In this embodiment, unlike in the first embodiment, the reaction vessel 101A has no waste liquid holes extending from the recess portions 115 to the lower surface. For this reason, as is obvious from comparison between FIGS. 12 and 28, there is no need to provide various elements (e.g., the suction pumps 135, electromagnetic valves 128, and waste liquid tanks 136 in FIG. 12) necessary for the discharge of a fluid using waste liquid holes. This leads to reduction in the size and power consumption of the examination apparatus.

The reaction vessel 101A of this embodiment has no waste liquid hole, and hence the volume of the channel inside it is smaller accordingly. For this reason, the transfer responsiveness of a fluid (e.g., a nucleic acid sample solution) improves with respect to pressure transfer by the syringe piston pump. This enables the examination time to be shortened.

In addition, since the reaction vessel 101A has no waste liquid holes, there are no channels between the recess portions 115 and the liquid storage holes 117, and the transfer amounts of fluid passing through the channels are made more stable. This makes it possible to perform examination with higher accuracy.

This embodiment may be variously modified and changed. For example, the washing water 137 may be simultaneously supplied from the cleaning nozzle 214 and from the syringe piston pump 131. In this case, a larger amount of fresh washing water can be made to pass through the porous portion 110 within a shorter period of time. That is, the supply time for the washing water 137 can be shortened. This enables the examination time to be shortened.

### Ninth Embodiment

This embodiment is directed to an examination apparatus in which the arrangement of the syringe piston pump in the eighth embodiment is changed to transfer fluid by air as in the fourth embodiment. In other words, like the eighth embodiment, this embodiment is directed to an examination apparatus which includes a reaction vessel obtained by omitting the waste liquid holes from the reaction vessel of the fourth embodiment, and a reaction stage suitably corresponding to the reaction vessel.

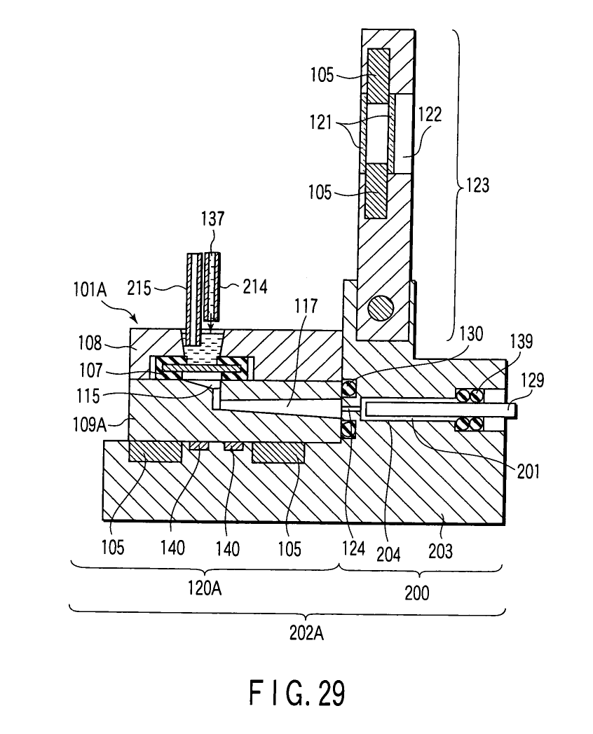

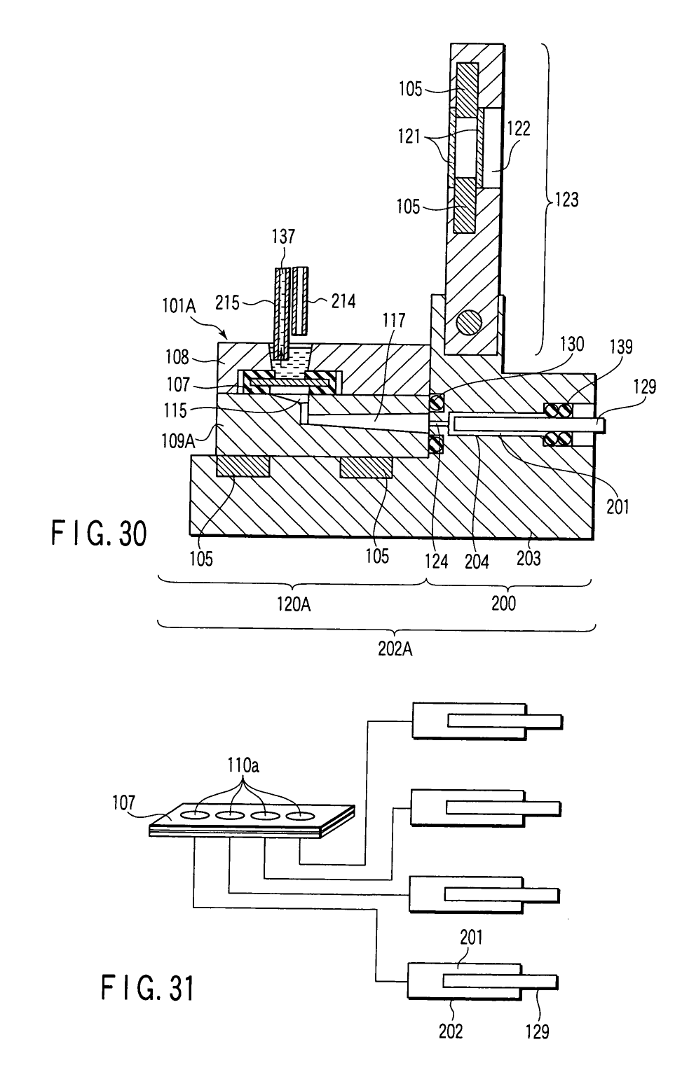

As shown FIGS. 29 and 30, a reaction stage 202A in this embodiment includes a base portion 120A on which a reaction vessel 101A is placed, a cover portion 123 which can open and close with respect to the base portion 120A, and a syringe piston pump unit 200 which is a fluid transfer mechanism for transferring a fluid with respect to the reaction vessel 101A.

The arrangements of the reaction vessel 101A and base portion 120A are the same as those in the eighth embodiment. The arrangement of the cover portion 123 is the same as that in the first embodiment. The arrangement of the syringe piston pump unit 200 is the same as that in the fourth embodiment.

That is, the syringe piston pump unit 200 includes four transfer channels 124 which are fluidally communicated with liquid storage holes 117 of the reaction vessel 101A, respectively, and four syringe piston pumps 203 which are fluidally in communication with the transfer channels 124, respectively, and fluidally communicated with the liquid storage holes 117 of the reaction vessel 101A, respectively.

Each syringe piston pump 203 includes a syringe 204, a piston 129 which can move in the syringe 204, and a seal 139 which is a seal for keeping the syringe 204 and the piston 129 in an airtight state.

With the suction and discharge of air 201 upon movement of the piston 129, the fluids (the nucleic acid sample solution and cleaning fluid) in the reaction vessel 101A supported on the reaction stage 202A are transferred.

The gene examination apparatus of this embodiment includes cleaning nozzles 214 which supply washing water 137 to the reaction vessel 101A from above and waste liquid nozzles 215 which remove the washing water 137 from the reaction vessel 101A, as in the eighth embodiment.

As in the fourth embodiment, the reaction vessel 101A is supported on the reaction stage 202A, each liquid storage hole 117 is fluidally communicated with the syringe piston pump 203. A hybridization reaction is performed in the same manner as in the fourth embodiment.

After the hybridization reaction, as shown in FIG. 29, for example, the washing water 137 is supplied from each cleaning nozzle 214 into a tapered opening portion 113 of an upper housing 108. With the sucking operation of the syringe piston pump 203, the washing water 137 in the opening portion 113 passes through the DNA microarray and moves in the liquid storage hole 117. Thereafter, with the discharging operation of the syringe piston pump 203, the washing water 137 in the liquid storage hole 117 passes through the DNA microarray and moves into the opening portion 113.

After the sucking operation and discharging operation of the syringe piston pump 203 are properly repeated, the washing water 137 in the opening portion 113 is sucked by the waste liquid nozzle 215 and removed, as shown in FIG. 30. This removes foreign substances existing on the DNA microarray, together with the washing water 137.

If the washing water 137 left on the DNA microarray influences an examination result after suction by the waste liquid nozzle 215, the washing water 137 is preferably sucked into the liquid storage hole 117 by a syringe piston pump 203.

In this embodiment, since the washing water 137 is sucked by the waste liquid nozzle 215 placed above the DNA microarray, foreign substances existing on the DNA microarray are removed, together with the washing water 137.

With respect to the eighth embodiment, this embodiment has the same merit as that which the fourth embodiment has with respect to the first embodiment. Since this merit has been described in detail in the fourth embodiment, a description thereof will be omitted.

In addition, with respect to the fourth embodiment, this embodiment has the same merit as that which the eighth embodiment has with respect to the first embodiment.

That is, in this embodiment, unlike in the fourth embodiment, the reaction vessel 101A has no liquid holes extending from the recess portions 115 to the lower surface. For this reason, as is obvious from comparison between FIGS. 20 and 31, there is no need to provide various elements (e.g., the suction pumps 135, electromagnetic valves 128, and waste liquid tanks 136 in FIG. 20) necessary for the discharge of a fluid using waste liquid holes. This leads to reduction in the size and power consumption of the examination apparatus.

The reaction vessel 101A of this embodiment has no waste liquid hole, and hence the volume of the channel inside it is smaller accordingly. For this reason, the transfer responsiveness of a fluid (e.g., a nucleic acid sample solution) improves with respect to pressure transfer by the syringe piston pump. This enables the examination time to be shortened.

In addition, since the reaction vessel 101A has no waste liquid holes, there are no channels between the recess portions 115 and the liquid storage holes 117, and the transfer amounts of fluid passing through the channels are made more stable. This makes it possible to perform examination with higher accuracy.

In addition, since the pressure transfer unit using the syringe piston pump 203 is located near each liquid storage hole 117 of the reaction vessel 101A, the fluid transfer responsiveness and the stability of transfer amounts further improve. This enables the examination time to be further shortened and the examination accuracy to be further improved.

Furthermore, since each syringe piston pump has no structure to be filled with a cleaning fluid, further reductions in the size and power consumption of the apparatus can be realized.

### Tenth Embodiment

This embodiment is directed to a gene examination apparatus including a reaction stage which directly supports a slide chip.

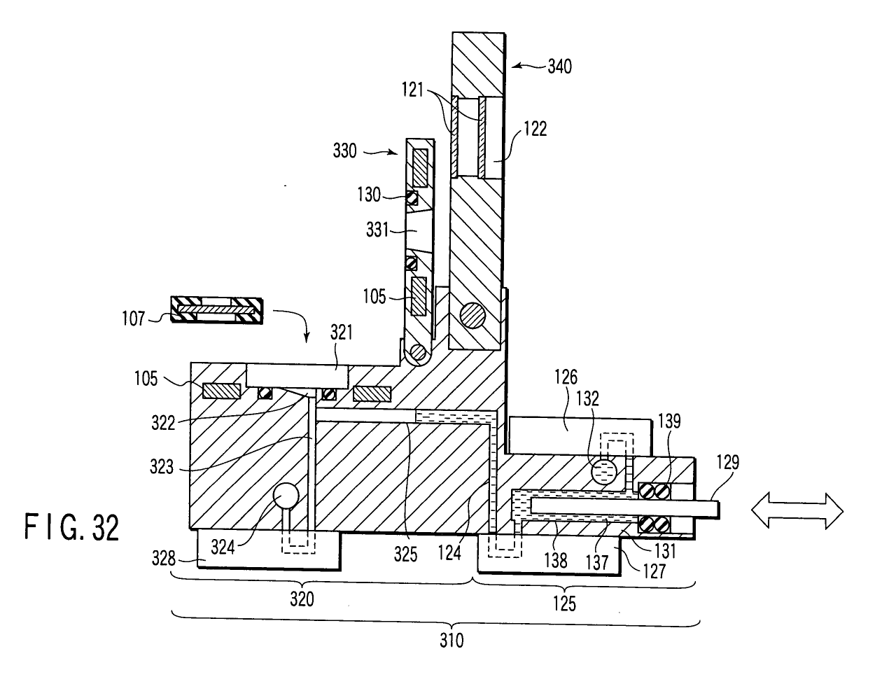

As shown in FIGS. 32 to 36, the gene examination apparatus according to this embodiment includes a reaction stage 310 for supporting a slide chip 107 having DNA microarrays and promoting reactions with the DNA microarrays.

The reaction stage 310 includes a base portion 320 having a recess portion 321 which can house the slide chip 107, an inner cover portion 330 which can open and close with respect to the base portion 320, an outer cover portion 340 which can open and close with respect to the base portion 320, and a syringe piston pump unit 125.

Both the inner cover portion 330 and the outer cover portion 340 rotate about their axes to open and close with respect to the base portion 320. The opening/closing operation of the inner cover portion 330 and outer cover portion 340 may be manually or electrically performed.

The inner cover portion 330 includes four tapered opening portions 331 formed at positions corresponding to four DNA microarrays 110a of the slide chip 107. The inner space of each opening portion 331 can stored a fluid containing a nucleic acid sample to be supplied to the DNA microarray 110a when the inner cover portion 330 is closed.

The base portion 320 has four recess portions 322 which are formed in the bottom surface of the recess portion 321 housing the slide chip 107 and have inclined bottom surfaces. The four recess portions 322 are formed at positions corresponding to the four DNA microarrays 110a of the slide chip 107 housed in the recess portion 321. The base portion 320 further includes four discharge channels 323 for waste liquid which extend from the lowermost portions of the recess portions 322, a waste liquid collecting pipe 324 with which the four discharge channels 323 are fluidally in communication, and four electromagnetic valves 328 for opening and closing the discharge channels 323.

Each electromagnetic valve 328 is provided on the lower surface of the base portion 320. The discharge channel 323 extends from the recess portion 322 to the waste liquid collecting pipe 324 through the electromagnetic valve 328. That is, the discharge channel 323 passes through the base portion 320, enters the base portion 320 again through the electromagnetic valve 328, and terminates at the waste liquid collecting pipe 324.

The base portion 320 further has a liquid storage hole 325 extending midway from each discharge channel 323 to a side. The liquid storage hole 325 can store a fluid containing the nucleic acid sample to be supplied to the DNA microarray 110a. The liquid storage hole 325 is fluidally in communication with the syringe piston pump unit 125.

The arrangement of the syringe piston pump unit 125 is the same as that in the first embodiment. That is, the syringe piston pump unit 125 includes four transfer channels 124 which are fluidally in communication with the liquid storage holes 325 in the base portion 320, four syringe piston pumps 131 which are fluidally in communication with the transfer channels 124, respectively, and electromagnetic valves 127 for opening and closing the transfer channels 124.

The outer cover portion 340 includes a microscope observation hole 122 which allows observation of the DNA microarray 110a with a microscope 102, and two optically transparent cover glasses 121 which close the microscope observation hole 122. The two cover glasses 121 are spaced apart from each other.

The base portion 320 has circular grooves formed in the bottom surface of the recess portion 321 housing the slide chip 107 so as to surround the recess portions 322. O-rings 130 are placed in the grooves. The inner cover portion 330 has circular grooves in the lower surface facing the closed slide chip 107 so as to surround the opening portions 331. O-ring 130 are placed in the grooves.

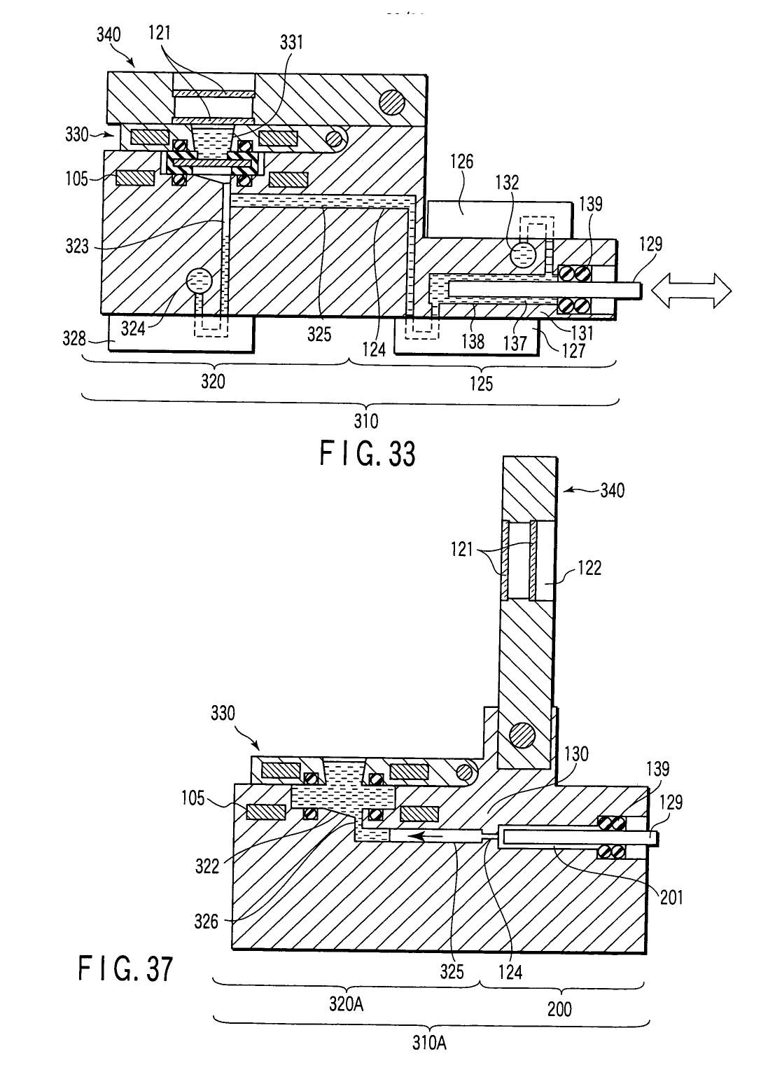

As shown in FIG. 33, when closed, the inner cover portion 330 supports the slide chip 107 in cooperation with the base portion 320 by holding it between them. That is, the base portion 320 and the inner cover portion 330 constitute a slide chip support mechanism which supports the slide chip 107.

The O-rings 130 provided for the base portion 320 and inner cover portion 330 respectively function to keep the liquid tightness between the base portion 320 and the slide chip 107 and between the inner cover portion 330 and the slide chip 107.

The seals which keep liquid tightness between the base portion 320 and the slide chip 107 and between the inner cover portion 330 and the slide chip 107 in this manner are not limited to O-rings, and other arbitrary proper seals, e.g., packings or Teflon seals, can be used. In addition, seals may be provided on the slide chip 107 side as long as the liquid tightness between the base portion 320 and the slide chip 107 and between the inner cover portion 330 and the slide chip 107 is kept.

The reaction stage 310 further includes a temperature adjusting mechanism similar to that in the first embodiment. As shown in FIGS. 32 to 36, therefore, the base portion 320 and the inner cover portion 330 include plate-like heaters 105 for heating the slide chip 107.

In order to efficiently heat the slide chip 107, the plate-like heaters 105 in the base portion 320 are preferably provided near the recess portion 321 housing the slide chip 107. In addition, the plate-like heaters 105 in the inner cover portion 330 are preferably provided at such positions that position near the recess portion 321 of the base portion 320 which houses the slide chip 107 when closed as shown in FIG. 33.

In addition, although not shown, the reaction stage 310 may further include cooling means comprising Peltier elements and the like as in the first embodiment.

The gene examining operation of this embodiment will be described below.

As shown in FIG. 32, first of all, while both the outer cover portion 340 and inner cover portion 330 are open, the slide chip 107 is placed in the recess portion 321 of the base portion 320. The inner cover portion 330 is then closed, and the slide chip 107 is caught between the base portion 320 and the inner cover portion 330 to be held. In this state, the slide chip 107, base portion 320, and inner cover portion 330 are kept liquid tight. Thereafter, the nucleic acid sample solution 114 is supplied into the opening portion 331 of the inner cover portion 330, and then the outer cover portion 340 is closed, as shown in FIG. 33.

Subsequently, as in the first embodiment, the nucleic acid sample solution 114 is caused to pass through the porous portion 110 by the syringe piston pump 131 while the slide chip 107 and the nucleic acid sample solution 114 are heated to a proper temperature by the heaters 105, so that a hybridization reaction can be finished within a relatively short period of time.

After the hybridization reaction, the slide chip 107 is examined with an optical microscope or the like. After the examination, the outer cover portion 340 and the inner cover portion 330 are opened, and the slide chip 107 having undergone the examination is removed.

Subsequently, for the next examination, another slide chip 107 is mounted on the reaction stage 310. Alternatively, examination is terminated. When examination is terminated, a portion of the reaction stage 310 with which the nucleic acid sample solution has come into contact is cleaned. When another slide chip 107 is to be examined, a portion of the reaction stage 310 with which the nucleic acid sample solution has come into contact is cleaned as needed to prevent contamination before another slide chip 107 is mounted.

If another slide chip 107 to be examined next is identical to the previously examined slide chip 107, since there is no change of contamination, such cleaning operation may be omitted.

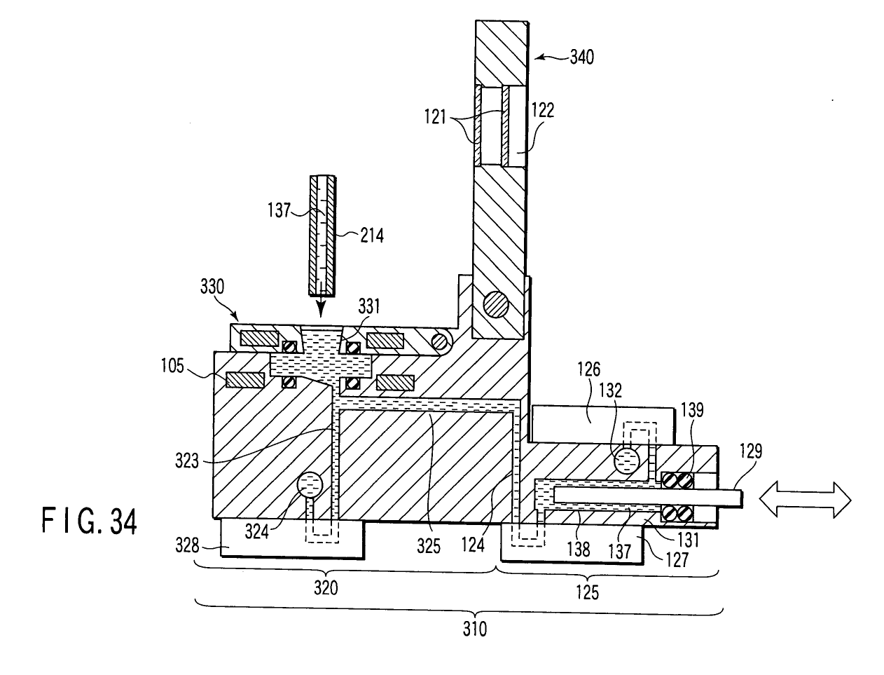

Cleaning operation will be described next. While the slide chip 107 is removed from the base portion 320, the inner cover portion 330 is closed as shown in FIG. 34, and washing water 137 is supplied into the inner space of the opening portion 331 of the inner cover portion 330 by a cleaning nozzle 214.

Subsequently, with the sucking operation of the syringe piston pump 131, the washing water 137 in the opening portion 331 passes through the DNA microarray and moves into the liquid storage hole 325. With the discharging operation of the syringe piston pump 131, the washing water 137 in the liquid storage hole 325 then passes through the DNA microarray and moves into the opening portion 331.

After the sucking operation and discharging operation of the syringe piston pump 131 are properly repeated, the washing water 137 is sucked by the suction pump 135. With this operation, the washing water 137 is discharged from the reaction stage 310 through the discharge channel 323.

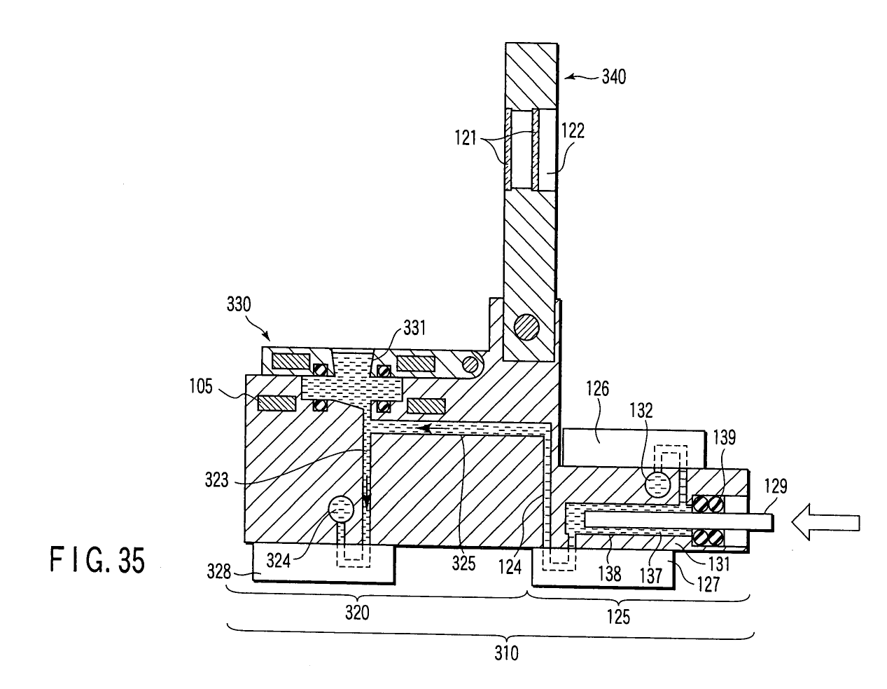

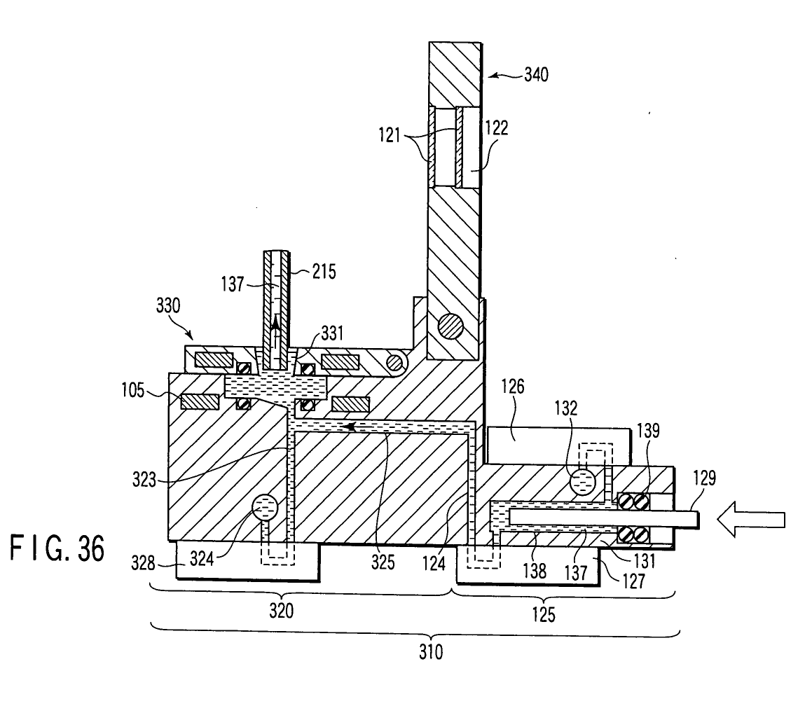

Although FIG. 34 has exemplified the case wherein the washing water 137 is supplied from the cleaning nozzle 214, the washing water 137 may also be supplied from the syringe piston pump 131, as shown in FIG. 35. Thereafter, as shown in FIG. 36, the washing water 137 is supplied into the opening portion 331 to such a degree that the washing water is stored therein, and an excess of the washing water 137 is discharged from the reaction stage 310 through the discharge channel 323 by the suction pump 135.

In addition, a technique to be used to discharge the washing water 137 is not limited to the technique using the discharge channel 323. For example, as shown in FIG. 36, a waste liquid nozzle 215 may be placed in the opening portion 331 to suck the washing water 137 by using the waste liquid nozzle 215.

In the examination apparatus of this embodiment, since changing operation is performed on a slide chip basis. Since each slide chip 107 is smaller in volume than the reaction vessel housing the slide chip 107, the transfer speed of heat to a solid support increases as compared with the examination apparatus in which changing operation is performed on a reaction vessel basis. This enables the examination time to be shortened. In addition, heat conduction irregularity decreases. Furthermore, more slide chips can be placed within a predetermined limited area of the examination apparatus. This makes it possible to perform more examinations at once.

This embodiment can be variously modified and changed. For example, in this embodiment, both the inner cover portion 330 and the outer cover portion 340 are configured to open and close with respect to the base portion 320 by rotating about the respective axes. However, they may be configured to open and close with respect to the base portion 320 by moving in the horizontal direction.

### Eleventh Embodiment

This embodiment is directed to another gene examination apparatus including a reaction stage which directly supports a slide chip. In the gene examination apparatus according to this embodiment, the discharge channels and related elements are omitted from the base portion in the tenth embodiment, and the arrangement of the syringe piston pump unit is changed such that a fluid is transferred by air.

As shown in FIG. 37, a reaction stage 310A in this embodiment includes a reaction stage 310A, a base portion 320A, an inner cover portion 330 which can open and close with respect to the base portion 320A, an outer cover portion 340 which can open and close with respect to the base portion 320A, and a syringe piston pump unit 200.

The arrangements of the inner cover portion 330 and outer cover portion 340 are the same as those in the tenth embodiment.

The base portion 320A in this embodiment has an arrangement obtained by omitting the discharge channels 323, the waste liquid collecting pipe 324, and the electromagnetic valves 328 from the base portion 320 in the tenth embodiment. For this reason, a recess portion 322 and a liquid storage hole 325 are fluidally in communication with each other through a vertical hole portion 326 extending between the lowermost portion of the recess portion 322 and an end portion of the liquid storage hole 325.

The arrangement of the syringe piston pump unit 200 is almost the same as that in the fourth embodiment.

In this embodiment, four syringe piston pumps 203 of the syringe piston pump unit 200 are fluidally in communication with the liquid storage holes 325 of the base portion 320A, respectively, through transfer channels 124.

In the examination apparatus of this embodiment, a hybridization reaction is performed in the same manner as in the tenth embodiment except that a nucleic acid sample solution is transferred by air using the syringe piston pump unit 200.

In the examination apparatus of this embodiment, cleaning operation is performed in almost the same manner as in the tenth embodiment. First of all, while a slide chip 107 is removed from the base portion 320, the inner cover portion 330 is closed, and washing water 137 is supplied from a cleaning nozzle 214 (see FIG. 34) into the internal space of an opening portion 331 of the inner cover portion 330.

With the sucking operation of the syringe piston pump 203, the washing water 137 in the internal space of the opening portion 331 passes through the DNA microarray and moves into the liquid storage hole 325. Thereafter, with the discharging operation of the syringe piston pump 203, the washing water 137 in the liquid storage hole 325 passes through the DNA microarray and moves into the internal space of the opening portion 331.

After the sucking operation and discharging operation of the syringe piston pump 203 are properly repeated, the washing water 137 in an opening portion 113 is sucked by a waste liquid nozzle 215 (see FIG. 36) to be removed. This removes foreign substances existing on the DNA microarray, together with the washing water 137.

As in the tenth embodiment, in the examination apparatus of this embodiment, since changing operation is performed for each slide chip 107, the transfer speed of heat to a solid support increases. Therefore, the examination time is shortened. In addition, heat conduction irregularity decreases. Furthermore, more slide chips can be placed within a predetermined limited area of the examination apparatus. This makes it possible to perform more examinations at once.

The merit in changing the syringe piston pump unit to the syringe piston pump unit 200 which transfers a liquid by air is the same as that in the fourth embodiment. The merit in omitting the discharge channels and the like from the base portion is the same as that in the eighth embodiment.

Although the embodiments of the present invention have been described with reference to the accompanying drawing, the present invention is not limited to these embodiments. The present invention can be variously modified and changed within the spirit and scope of the invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A biorelated substance examination apparatus for examining a reaction of a biorelated substance using a solid support which allows a fluid to pass therethrough, comprising:
a reaction stage which supports a reaction vessel which contains the solid support and promotes a reaction of the solid-state phase; and
a microscope which optically observes the solid support,
the reaction stage including a fluid transfer mechanism which transfers a fluid with respect to the reaction vessel and a temperature adjusting mechanism which adjusts a temperature of the solid support in the reaction vessel.

2. A biorelated substance examination apparatus according to claim 1, wherein the temperature adjusting mechanism includes heating instrument to raise the temperature of the solid support.

3. A biorelated substance examination apparatus according to claim 2, wherein the temperature adjusting mechanism also includes cooling instrument to lower the temperature of the solid support.

4. A biorelated substance examination apparatus according to claim 1, which further comprises a hood to cover the reaction stage and inserting and drawing mechanism which inserts and draws out the reaction stage into and from the hood, and in which the inserting and drawing means draws out the reaction stage from the hood when a temperature of the reaction stage becomes not more than a predetermined temperature.

5. A biorelated substance examination apparatus according to claim 2, further comprising a cooling mechanism to lower the temperature of the solid support by forced convection, the cooling mechanism including a blower which feeds air to the reaction stage.

6. A biorelated substance examination apparatus according to claim 5, wherein the cooling mechanism further includes a Peltier element to cool air fed to the reaction stage.

7. A biorelated substance examination apparatus according to claim 1, wherein the solid support is supported in the reaction vessel.

8. A biorelated substance examination apparatus according to claim 7, wherein
the reaction vessel includes a liquid storage hole which is fluidally in communication with the solid support and adapted to store the fluid in the reaction vessel, and
the fluid transfer mechanism includes a transfer channel which is fluidally communicated with the liquid storage hole of the reaction vessel, and a transfer pump which is fluidally in communication with the transfer channel and transfers the fluid in the reaction vessel.

9. A biorelated substance examination apparatus according to claim 8, wherein
the reaction vessel further includes a waste liquid hole which is fluidally in communication with the solid support and discharges the fluid in the reaction vessel to outside,
the reaction stage further includes a discharge channel which is fluidally communicated with the waste liquid hole of the reaction vessel, and
the examination apparatus further includes a fluid discharge mechanism to discharge the fluid in the reaction vessel to outside through the discharge channel of the reaction stage.

10. A biorelated substance examination apparatus according to claim 8, wherein the transfer pump comprises a syringe piston pump.

11. A biorelated substance examination apparatus according to claim 8, wherein the transfer pump transfers the fluid in the reaction vessel by sucking a gas thereinto and discharging the gas therefrom.

12. A biorelated substance examination apparatus according to claim 8, wherein the fluid transfer mechanism further includes a pressure sensor to detect a pressure of the fluid in the transfer channel.

13. A biorelated substance examination apparatus according to claim 1, further comprising a nozzle to suck the fluid in the reaction vessel from an examination surface side of the solid support.

14. A biorelated substance examination apparatus according to claim 1, further comprising a nozzle to discharge the fluid in the reaction vessel from an examination surface side of the solid support.

15. A reaction stage for an apparatus which examines a reaction of a biorelated substance using a solid support which allows a fluid to pass therethrough, comprising:
a reaction vessel support mechanism which supports a reaction vessel which contains the solid support;
a fluid transfer mechanism to transfer a fluid with respect to the reaction vessel; and
a temperature adjusting mechanism to adjust a temperature of the solid support in the reaction vessel.

16. A reaction stage according to claim 15, wherein the temperature adjusting mechanism includes heating instrument to raise the temperature of the solid support.

17. A reaction stage according to claim 16, wherein the temperature adjusting mechanism also includes cooling instrument to lower the temperature of the solid support.

18. A reaction stage according to claim 15, wherein the solid support is supported in the reaction vessel.

19. A reaction stage according to claim 18, wherein
the reaction vessel includes a liquid storage hole which is fluidally in communication with the solid support and adapted to store the fluid in the reaction vessel, and
the fluid transfer mechanism includes a transfer channel which is fluidally communicated with the liquid storage hole of the reaction vessel, and a transfer pump which is fluidally in communication with the transfer channel and transfers the fluid in the reaction vessel.

20. A reaction stage according to claim 19, wherein
the reaction vessel further includes a waste liquid hole which is fluidally in communication with the solid support and discharges the fluid in the reaction vessel to outside, and
the reaction stage further includes a discharge channel which is fluidally communicated with the waste liquid hole of the reaction vessel.

21. A reaction stage according to claim 19, wherein the transfer pump comprises a syringe piston pump.

22. A reaction stage according to claim 19, wherein the transfer pump transfers the fluid in the reaction vessel by sucking a gas thereinto and discharging the gas therefrom.

23. A reaction stage according to claim 19, wherein the fluid transfer mechanism further includes a pressure sensor to detect a pressure of the fluid in the transfer channel.

24. A reaction stage for an apparatus which examines a reaction of a biorelated substance using a solid support which allows a fluid to pass therethrough, comprising:
a slide chip support mechanism which supports a slide chip containing the solid support;
a fluid transfer mechanism which transfers a fluid with respect to the slide chip; and
a temperature adjusting mechanism which adjusts a temperature of the solid support.

25. A reaction stage according to claim 24, wherein the temperature adjusting mechanism includes heating instrument to raise the temperature of the solid support.

26. A reaction stage according to claim 25, wherein the temperature adjusting mechanism also includes cooling instrument to lower the temperature of the solid support.

27. A reaction stage according to claim 24, wherein the slide chip support mechanism includes a base portion including a recess portion which can house the slide chip, and a cover portion which can open and close with respect to the base portion, and the cover portion includes an opening portion to store the fluid on the solid support.

28. A reaction stage according to claim 27, wherein the reaction stage further includes another cover portion which closes the opening portion of the cover portion so as to allow optical observation of the solid support.

29. A reaction stage according to claim 27, wherein
the base portion includes a liquid storage hole which is fluidally in communication with the solid support and adapted to store the fluid stored in an inner space of the opening portion of the cover portion, and
the fluid transfer mechanism includes a transfer channel fluidally in communication with the liquid storage hole of the base portion and a transfer pump which is fluidally in communication with the transfer channel and transfers the fluid.

30. A reaction stage according to claim 29, wherein the base portion further includes a discharge channel which is fluidally communicated with a solid support.

31. A reaction stage according to claim 29, wherein the transfer pump comprises a syringe piston pump.

32. A reaction stage according to claim 29, wherein the transfer pump transfers the fluid in the reaction stage by sucking a gas thereinto and discharging the gas therefrom.

33. A reaction stage according to claim 29, wherein the fluid transfer mechanism further includes a pressure sensor to detect a pressure of the fluid in the transfer channel.

34. A biorelated substance examination apparatus for examining a reaction of a biorelated substance using a solid support which allows a fluid to pass therethrough, comprising:
a reaction stage for supporting a reaction vessel which contains the solid support and promoting a reaction of the solid-state phase; and
a microscope for optically observing the solid support,
the reaction stage including a fluid transfer mechanism for transferring a fluid with respect to the reaction vessel and a temperature adjusting mechanism for adjusting a temperature of the solid support in the reaction vessel.

35. A biorelated substance examination apparatus according to claim 34, wherein the temperature adjusting mechanism includes heating means for raising the temperature of the solid support.

36. A biorelated substance examination apparatus according to claim 35, wherein the temperature adjusting mechanism also includes cooling means for lowering the temperature of the solid support.

37. A biorelated substance examination apparatus according to claim 34, which further comprises a hood for covering the reaction stage and inserting and drawing means which inserts and draws out the reaction stage into and from the hood, and in which the inserting and drawing means draws out the reaction stage from the hood when a temperature of the reaction stage becomes not more than a predetermined temperature.

38. A biorelated substance examination apparatus according to claim 35, further comprising a cooling mechanism for lowering the temperature of the solid support by forced convection, the cooling mechanism including a blower which feeds air to the reaction stage.

39. A biorelated substance examination apparatus according to claim 38, wherein the cooling mechanism further includes a Peltier element for cooling air fed to the reaction stage.

40. A biorelated substance examination apparatus according to claim 34, wherein the solid support is supported in the reaction vessel.

41. A biorelated substance examination apparatus according to claim 40, wherein
the reaction vessel includes a liquid storage hole which is fluidally in communication with the solid support and adapted to store the fluid in the reaction vessel, and
the fluid transfer mechanism includes a transfer channel which is fluidally communicated with the liquid storage hole of the reaction vessel, and a transfer pump, which is fluidally in communication with the transfer channel, for transferring the fluid in the reaction vessel.

42. A biorelated substance examination apparatus according to claim 41, wherein
the reaction vessel further includes a waste liquid hole, which is fluidally in communication with the solid support, for discharging the fluid in the reaction vessel to outside,
the reaction stage further includes a discharge channel which is fluidally communicated with the waste liquid hole of the reaction vessel, and
the examination apparatus further includes a fluid discharge mechanism for discharging the fluid in the reaction vessel to outside through the discharge channel of the reaction stage.

43. A biorelated substance examination apparatus according to claim 41, wherein the transfer pump comprises a syringe piston pump.

44. A biorelated substance examination apparatus according to claim 41, wherein the transfer pump transfers the fluid in the reaction vessel by sucking a gas thereinto and discharging the gas therefrom.

45. A biorelated substance examination apparatus according to claim 41, wherein the fluid transfer mechanism further includes a pressure sensor for detecting a pressure of the fluid in the transfer channel.

46. A biorelated substance examination apparatus according to claim 34, further comprising a nozzle for sucking the fluid in the reaction vessel from an examination surface side of the solid support.

47. A biorelated substance examination apparatus according to claim 34, further comprising a nozzle for discharging the fluid in the reaction vessel from an examination surface side of the solid support.

48. A reaction stage for an apparatus which examines a reaction of a biorelated substance using a solid support which allows a fluid to pass therethrough, comprising:
a reaction vessel support mechanism which supports a reaction vessel which contains the solid support;
a fluid transfer mechanism for transferring a fluid with respect to the reaction vessel; and
a temperature adjusting mechanism for adjusting a temperature of the solid support in the reaction vessel.

49. A reaction stage according to claim 48, wherein the temperature adjusting mechanism includes heating means for raising the temperature of the solid support.

50. A reaction stage according to claim 49, wherein the temperature adjusting mechanism also includes cooling means for lowering the temperature of the solid support.

51. A reaction stage according to claim 48, wherein the solid support is supported in the reaction vessel.

52. A reaction stage according to claim 51, wherein
the reaction vessel includes a liquid storage hole which is fluidally in communication with the solid support and adapted to store the fluid in the reaction vessel, and
the fluid transfer mechanism includes a transfer channel which is fluidally communicated with the liquid storage hole of the reaction vessel, and a transfer pump, which is fluidally in communication with the transfer channel, for transferring the fluid in the reaction vessel.

53. A reaction stage according to claim 52, wherein
the reaction vessel further includes a waste liquid hole, which is fluidally in communication with the solid support, for discharging the fluid in the reaction vessel to outside, and
the reaction stage further includes a discharge channel which is fluidally communicated with the waste liquid hole of the reaction vessel.

54. A reaction stage according to claim 52, wherein the transfer pump comprises a syringe piston pump.

55. A reaction stage according to claim 52, wherein the transfer pump transfers the fluid in the reaction vessel by sucking a gas thereinto and discharging the gas therefrom.

56. A reaction stage according to claim 52, wherein the fluid transfer mechanism further includes a pressure sensor for detecting a pressure of the fluid in the transfer channel.

57. A reaction stage for an apparatus which examines a reaction of a biorelated substance using a solid support which allows a fluid to pass therethrough, comprising:
a slide chip support mechanism which supports a slide chip containing the solid support;
a fluid transfer mechanism for transferring a fluid with respect to the slide chip; and
a temperature adjusting mechanism for adjusting a temperature of the solid support.

58. A reaction stage according to claim 57, wherein the temperature adjusting mechanism includes heating means for raising the temperature of the solid support.

59. A reaction stage according to claim 58, wherein the temperature adjusting mechanism also includes cooling means for lowering the temperature of the solid support.

60. A reaction stage according to claim 57, wherein the slide chip support mechanism includes a base portion including a recess portion which can house the slide chip, and a cover portion which can open and close with respect to the base portion, and the cover portion includes an opening portion for storing the fluid on the solid support.

61. A reaction stage according to claim 60, wherein the reaction stage further includes another cover portion which closes the opening portion of the cover portion so as to allow optical observation of the solid support.

62. A reaction stage according to claim 60, wherein
the base portion includes a liquid storage hole which is fluidally in communication with the solid support and adapted to store the fluid stored in an inner space of the opening portion of the cover portion, and
the fluid transfer mechanism includes a transfer channel fluidally in communication with the liquid storage hole of the base portion and a transfer pump, which is fluidally in communication with the transfer channel, for transferring the fluid.

63. A reaction stage according to claim 62, wherein the base portion further includes a discharge channel which is fluidally communicated with a solid support.

64. A reaction stage according to claim 62, wherein the transfer pump comprises a syringe piston pump.

65. A reaction stage according to claim 62, wherein the transfer pump transfers the fluid in the reaction stage by sucking a gas thereinto and discharging the gas therefrom.

66. A reaction stage according to claim 62, wherein the fluid transfer mechanism further includes a pressure sensor for detecting a pressure of the fluid in the transfer channel.
